(19) United States
(12) Patent Application Publication
(10) Pub. No.: US 2009/0053268 A1
(43) Pub. Date: Feb. 26, 2009

(54) NANOPARTICLE MODIFIED LUBRICANTS AND WAXES WITH ENHANCED PROPERTIES

(76) Inventors: Juan J. DePablo, Madison, WI (US); Mary Jo Biddy, Warrenville, IL (US)

Correspondence Address: GREENLEE WINNER AND SULLIVAN P C, 4875 PEARL EAST CIRCLE, SUITE 200, BOULDER, CO, 80301, US

(21) Appl. No.: 11/843,303

(22) Filed: Aug. 22, 2007

(51) Int. Cl. C11C 5/00; A61K 9/10; A61K 47/44; C10M 169/04; C08L 91/06; C09G 1/08; A61K 8/92; A61K 8/04

(52) U.S. Cl. 424/401; 508/491; 44/275; 514/786; 424/484; 508/136; 508/154; 508/116; 106/10; 106/270; 554/115

(57) ABSTRACT

The present invention provides compositions and products, such as waxes and lubricants, comprising a plurality of nanoparticles dispersed in a continuous phase comprising a vegetable oil derived material, such as one or more vegetable oils or a synthetic product derived from one or more vegetable oils. Incorporation of nanoparticles in the present compositions is beneficial for providing mechanical, thermal and/or chemical properties useful for a selected product or product application. In some compositions of the present invention, for example, incorporation of the nanoparticle component provides compositions derived from one or more vegetable oils exhibiting enhanced mechanical stability, hardness, viscosity, thermal stability and mechanical strength.

### STATEMENT REGARDING FEDERALLY SPONSORED RESEARCH OR DEVELOPMENT

This invention was made with government support awarded by the following agencies: Department of Energy DE-FG02-97ER25308. The United States government has certain rights in the invention.

### CROSS-REFERENCE TO RELATED APPLICATIONS

Not applicable.

### BACKGROUND OF INVENTION

Advances in the development of nanocomposite materials have made a significant impact on a number of important technical fields including sensing, biotechnology, electronics, mechanical and structural additives, catalysis and optics. These advances are largely attributable to ongoing research directed to discovering new synthetic routes for making useful nanocomposite materials and characterizing the structural and functional properties of these materials. Nanocomposite materials exhibit structural and/or compositional inhomogeneities on a submicron scale, and often comprise a dispersed phase comprising nanoparticles provided in a liquid or solid continuous phase. The properties of nanocomposite materials may be dependent on a number of variables including the composition of the nanoparticles and continuous phase, and the morphology, physical dimensions, concentration, and interfacial characteristics of the dispersed nanoparticles. In many of these systems, the presence of dispersed nanoparticles gives rise to complex intermolecular interactions providing a molecular scale arrangement of the nanocomposite material resulting in useful mechanical, optical, electric, magnetic, and/or chemical properties.

The development of nontoxic, biodegradable and environmentally safe materials is a major area of research for which nanocomposite materials have potential to play an important role. With the rising costs of petroleum and concerns about the toxicity of petroleum based products to the environment, substantial interest is growing in developing alternatives to petroleum-base lubricants and waxes. Biodegradable lubricants and waxes based on plant and animal materials, such as canola oil, soy bean oil, corn oil and soy wax, show promise and have been used in the lubricant market, to a small extent, for some time. Many of these biodegradable alternatives, however, are currently more expensive to manufacture than petroleum-based products and also tend to have inferior physical and chemical properties as compared to petroleum based materials. Vegetable oil triglycerides, for example, are an abundant and promising class of materials that have great potential for use in a range of biodegradable products. The use of these materials in lubricating oils, however, is currently limited due to their susceptibility to oxidative degradation and their poor low temperature physical properties, such as their relatively high pour point temperatures as compared to comparable petroleum based materials.

As a result of the well recognized potential benefits provided by natural oil derived biodegradable materials, substantial research is currently directed toward developing cost effective strategies to improve the physical characteristics of these materials for a range of useful applications. Research in the field of biodegradable lubricants and waxes based on plant and animal materials, for example, is motivated, in part, by the need for additives for these materials capable of improving oxidative and thermal stability so as to extend their useful lifetimes and performance capabilities. The development of nanocomposite biodegradable materials via the incorporation of nanomaterial additives to vegetable oil derived materials is one strategy that is currently identified as a potentially cost effective route to the enhancement of physical and chemical properties of these materials.

International Publication No. WO 2006/076728 discloses the use of various nanomaterial additives as a viscosity modifier and thermal conductivity improver for lubricating oil compositions, including petroleum derived oils and vegetable oils. Nanoparticle additives including carbon nanostructures (e.g., nanotubes), metal particles, solid lubricants (e.g., molybdenum disulfide) and abrasive particles (e.g., aluminum oxide, silicon carbide) having physical dimension ranging from 1 to 200 nm are described. Use of nanoparticle additives in gear oils is characterized in this reference as providing a higher viscosity index, higher shear stability and improved thermal conductivity as compared to conventional gear oils without a nanoparticle component. In addition, a reduction in the coefficient of friction is also reported for some of the disclosed nanoparticle containing lubricant materials.

U.S. Pat. No. 6,878,676 discloses lubricant compositions containing molybdenum sulfide nanosized particles and related methods of making molybdenum sulfide nanosized particle-containing lubricants. Lubricant compositions containing dispersed molybdenum nanoparticles having diameters of 1 to 100 nm and weight percents ranging from 0.5-30% are described. In addition, the use of surface modified molybdenum sulfide nanosized particles with specific ligands is reported as useful for preventing nanoparticle coagulation, enhancing stability and increasing solubility.

International Publication No. WO 2005/0124504 discloses lubricant compositions having a nanomaterial additive and a dispersing agent. Nanomaterial additives described include carbon nanomaterials, such as carbon nanotubes, carbon nanofibrils and carbon nanoparticles, having physical dimensions less than 500 nanomaters in diameter. Lubricant additives and dispersing agents are reported to provide an enhancement of long-term stability and a high viscosity index. The reference also discloses control of nanomaterial additive size and dispersing chemistry so as to provide a desired viscosity and thermal conductivity.

U.S. Pat. No. 6,783,746 discloses methods of preparing stable dispersions of carbon nanotubes in various materials, including synthetic oils and vegetable oils, for changing the physical and chemical properties of liquids. The disclosed methods include steps of dissolving an appropriate dispersant in a liquid and adding carbon nanotubes via agitation and/or ultrasonication. Improvements in heat transfer, electrical properties, viscosity and lubricity are reported using the disclosed methods and compositions.

While advances in modulating the properties of lubricants via incorporation of nanomaterials have been reported, significantly less attention has been directed toward developing nanomaterials strategies for enhancing the properties of waxes derived from natural materials. U.S. Patent Publication 2005/0065238 discloses wax-containing compositions and oil containing compositions having encapsulated nanoparticles for uses as textile sizing materials and fiber coating materials. U.S. Patent Publication 2005/0155,515 discloses a water in oil emulsion wax containing aluminum oxide particles having particles sizes of 20 microns or less for use as a polishing agent.

Conventional waxes derived from vegetable oil-based materials, are known to exhibit a number of significant deficiencies, such as cracking and air pocket formation, that make them unsuitable for some applications. Candles made of conventional waxes derived from vegetable oils, for example, are known to exhibit problems relating to wax and wick performance, shortened burning time and limited product shelf life. Further, some conventional waxes derived from vegetable oils also exhibit mechanical properties, such as hardness and storage modulus, that are significantly less than petroleum-based waxes. These deficiencies current limit commercial implementation of waxes derived from natural materials for a range of applications such as manufacturing candles, vehicle and boat wax, pharmaceuticals, cleaning agents and cosmetics.

As will be understood from the foregoing, there currently exists a need in the art for methods and compositions for enhancing the physical and chemical properties of lubricants and waxes derived from natural materials, such as vegetable oils. Vegetable oil-based wax compositions are needed that exhibit enhanced mechanical properties, such as hardness and storage modulus. Vegetable oil-based wax compositions are needed that have physical and chemical properties useful for a variety of product applications. Vegetable oil derived compositions, such as waxes and lubricants, are needed that exhibit physical and chemical properties comparable to, or exceeding, those of petroleum-based materials.

### SUMMARY OF THE INVENTION

The present invention provides compositions and products, such as waxes and lubricants, comprising a plurality of nanoparticles dispersed in a continuous phase comprising a vegetable oil derived material, such as one or more vegetable oils or a synthetic product derived from one or more vegetable oils. A composition of this aspect of the present invention comprises a vegetable oil or synthetic product derived from a vegetable oil, and a plurality of nanoparticles dispersed in the vegetable oil or synthetic product derived from a vegetable oil, wherein the nanoparticles have an average cross-sectional dimension selected from the range of about 1 nanometer to about 100 nanometers, and wherein the nanoparticles comprise between about 1% and about 50% by mass of the composition. Embodiments of this aspect of the present invention include, but are not limited to, vegetable oil derived waxes and vegetable oil derived lubricants having a dispersed nanoparticle phase.

Incorporation of nanoparticles in the present compositions is beneficial for providing mechanical, thermal, optical and/or chemical properties useful for a selected product or product application. In some compositions of the present invention, for example, incorporation of the nanoparticle component provides compositions derived from one or more vegetable oils exhibiting enhanced mechanical stability, hardness, viscosity, thermal stability and mechanical strength. In some compositions of the present invention, for example, incorporation of the nanoparticle component provides wax compositions having enhanced optical properties relevant to exposure of the wax to light, such as ultraviolet light, relative to conventional waxes. Nanoparticle components of some aspects of the present invention have physical properties (e.g., morphology, physical dimensions, size distribution etc.), chemical properties (e.g. composition) and interfacial characteristics that give rise to intermolecular interactions providing a molecular scale arrangement of the nanocomposite material resulting in useful bulk phase mechanical and/or chemical properties. The invention includes products and articles of manufacture comprising the vegetable oil derived materials having dispersed nanoparticles providing enhanced physical and chemical properties.

The present invention also provides cost effective nanomaterials strategies for controlling the physical and chemical properties of natural oils and materials derived from natural oils. In these methods, nanoparticles are provided to a vegetable oil derived material, such as one or more natural vegetable oils or a synthetic product derived from one or more natural oils, in a manner to selectively adjust (or “tune”) one or more mechanical or thermal properties, such as hardness, durability, mechanical stability, viscosity, thermal stability and mechanical strength. In some embodiments, precise control of one or more selected physical and/or thermal properties is achieved by selection of the composition, physical dimensions, size distribution, concentration (e.g., percentage by mass), shape and/or morphology of the nanoparticle component provided to the vegetable oil derived material. The present invention also includes compositions and methods wherein a plurality of nanoparticle types are provided to a vegetable oil derived material, wherein the different nanoparticle types have different compositions, physical dimensions, shapes and/or morphologies selected to provide useful physical and chemical properties.

In an aspect, the present invention provides a wax containing composition comprising: a synthetic wax derived from one or more vegetable oils; and a plurality of nanoparticles dispersed in the synthetic wax. In an embodiment of this aspect, the dispersed nanoparticles have an average cross-sectional dimension selected from the range of about 1 nanometer to about 100 nanometers and the nanoparticles comprise between about 1% and about 50% by mass of the composition. Optionally, the wax containing composition of this embodiment may further comprise one or more additional additives, including, but not limited to, dispersants and/or stabilizers to enhance overall mechanical stability, thermal stability and/or shelf life. For example, compositions of the present invention may further comprise one or more surfactants for reducing or minimizing nanoparticle coagulation and/or settling. Other additives useful in the present compositions include one or more of suspension agents, a colorant, a fragrance and an emulsifying agent.

Synthetic waxes useful in this aspect of the present invention include, but are not limited to, triglyceride-based waxes derived from natural oils. In an embodiment, for example, a wax of the present invention comprises a triglyceride component that is greater than or equal to 20% by mass of the composition. Preferably for some applications a wax of the present invention comprises a triglyceride component having a concentration selected from the range of 20% to 80% by mass of the composition, and more preferably for some applications a triglyceride component having a concentration selected from the range of 20% to 50% by mass of the composition. Triglyceride-based waxes useful for certain compositions of the present invention comprise one or more hydrogenated or nonhydrogenated vegetable oils or are derived from one or more hydrogenated or nonhydrogenated vegetable oils. Exemplary vegetable oils for wax containing compositions of the present methods and compositions include, but are not limited to, soy bean oil; sunflower oil, corn oil, canola oil, castor oil, cottonseed oil, peanut oil, olive oil, sunflower oil, rapeseed oil, and safflower oil. Compositions and products of the present invention comprising soy bean wax or materials derived from soy bean wax are particularly attractive for some commercial applications given the abundance and low cost of this vegetable oil.

Selection of the compositions, physical dimensions, shapes, morphologies and concentrations (e.g., percentage by mass) of nanoparticles provided in the synthetic wax determines, in part, certain physical and chemical properties of compositions of this aspect of the present invention. In an embodiment providing compositions exhibiting enhanced hardness and storage modulus, the nanoparticles are spherical, have an average diameter selected from the range of about 10 nanometers to about 50 nanometers, and/or comprise between about 5% and about 30% by mass of the composition. In an embodiment of this aspect of the present invention, the nanoparticles have an average diameter of about 10 nanometers and comprise about 10% by mass of the compositions. Use of nanoparticles dispersed substantially uniformly throughout the synthetic wax (e.g., deviations within about 10% of an absolute uniform distribution) is beneficial for providing compositions having substantially uniform physical and/or chemical properties.

A variety of nanoparticles are useful in the present compositions and methods. Exemplary nanoparticles include, but are not limited to, (i) one or more silicon-containing nanoparticles selected from the group consisting of: silica nanoparticles, silicon carbide nanoparticles, and silicon nitride nanoparticles; (ii) one or more metal salt nanoparticles selected from the group consisting of: group 1 alkali metal hydroxide nanoparticles, group 1 alkali metal carbonate nanoparticles, group 1 alkali metal sulfate nanoparticles, group 1 alkali metal phosphate nanoparticles; group 1 alkali metal carboxylate nanoparticles, group 2 alkaline earth metal hydroxide nanoparticles, group 2 alkaline earth metal hydroxide carbonate nanoparticles, group 2 alkaline earth metal hydroxide sulfate nanoparticles, group 2 alkaline earth metal hydroxide phosphate nanoparticles; and group 2 alkaline earth metal hydroxide metal carboxylate nanoparticles; (iii) one or more transition metal-containing nanoparticles selected from the group consisting of transition metal oxide nanoparticles, transition metal carbide nanoparticles and transition metal nitride nanoparticles; (iv) one or more carbon nanoparticles selected from the group consisting of single walled carbon nanotubes, multiwalled carbon nanotubes, carbon nanorods, carbon nanofibers, and graphite particles; and (v) one or more metal nanoparticles. In an embodiment providing wax compositions exhibiting enhanced hardness and storage modulus, the nanoparticles are Mg(OH)₂, and/or silica (e.g., SiOₓ) nanoparticles. In an embodiment, the present invention provides compositions and methods wherein the nanoparticles are not encapsulated nanoparticles. The present invention includes, but is not limited to, compositions and methods wherein the nanoparticles are not encapsulated by one or more layers of polymer material

Compositions of this aspect of the invention provide a number of properties useful for a range of product applications. In an embodiment, for example, a wax containing composition of the present invention has a melting point temperature selected over the range of about 45 degrees Celsius to about 60 degrees Celsius. In an embodiment, for example, a wax containing composition of the present invention has a hardness selected over the range of 1.0 to 2.0 base HB (Brinell Hardness Test) 16/2 at 298K. The mechanical properties G′ and G″ were on both the order of magnitude of 10 to 100 Pa at temperatures above their melting points.

A significant benefit of compositions and methods of the present invention is that use of petroleum-based materials is reduced or entirely avoided. This aspect of the present invention is useful for reducing the toxicity of the present compositions and providing biodegradable compositions that are more environmentally safe than conventional petroleum-based materials. Further, the present compositions provide a renewable source of lubricants and waxes, as their vegetable oil derived components are themselves renewable. In an embodiment, for example, a composition of the present invention has less than about 10% by mass of a petroleum-derived chemical component, and preferably for some applications less than about 1% by mass of a petroleum-derived chemical component.

In another aspect, the present invention provides products and articles of manufacture comprising the compositions of the present invention. In an embodiment, for example, the present invention provides a nanoparticle modified wax, water in oil emulsion wax or spray wax comprising the present nanoparticle-containing compositions. In an embodiment, the present invention provides a candle, a coating wax, a polish for a vehicle, boat wax, a cosmetic wax, a pharmaceutical wax or a sealing wax comprising the present nanoparticle-containing compositions.

In another aspect, the present invention provides a method for enhancing at least one mechanical and/or optical property of a wax composition or a lubricant composition derived from one or more vegetable oils; the method comprising: (i) providing a synthetic wax derived from one or more vegetable oil or a synthetic lubricant derived from one or more vegetable oil; and (ii) dispersing in the synthetic wax or lubricant a plurality of nanoparticles thereby making the wax composition or the lubricant composition; the nanoparticles having an average cross-sectional dimension selected from the range of about 1 nanometers to about 100 nanometers; wherein the nanoparticles comprise between about 1% and about 50% by mass of the wax composition or the lubricant composition. Methods of this aspect of the present invention are useful for increasing the hardness, durability and/or solidity of a wax composition. Methods of this aspect of the present invention are useful for enhancing optical properties of a wax composition such as reflectance or extinction. Methods of this aspect of the present invention are useful for increasing the viscosity, thermal stability and/or shear stability of a lubricant composition. Optionally, in a method of the present invention the step of dispersing nanoparticles in the synthetic wax does not result in a decreasing the melting point of a synthetic wax.

In another aspect, the present invention provides a method for making a wax composition derived from one or more vegetable oils; the method comprising the steps of: (i) providing a synthetic wax derived from one or more vegetable oil; and (ii) dispersing in the synthetic wax a plurality of nanoparticles thereby making the wax composition; the nanoparticles having an average cross-sectional dimension selected from the range of about 1 nanometers to about 100 nanometers; wherein the nanoparticles comprise between about 1% and about 50% by mass of the wax composition, thereby making the wax composition derived from one or more vegetable oils.

In another aspect, the present invention provides a candle comprising: (i) a wax composition comprising synthetic wax derived from one or more vegetable oils; and a plurality of nanoparticles dispersed in the synthetic wax; the nanoparticles having an average cross-sectional dimension selected from the range of about 1 nanometer to about 100 nanometers; wherein the nanoparticles comprise between about 1% and about 50% by mass of the wax composition; and (ii) a wick disposed in the wax composition.

### BRIEF DESCRIPTION OF THE DRAWINGS

FIG. 1. Comparison of shear viscosity as a function of temperature for soybean oil-based lubricants. The viscosity of the oil was found to increase with increasing weight percentage of 10 nm silica nanoparticles.

FIG. 2. A comparison of the mechanical viscoelastic properties for the pure soybean oil and the soybean oil with 10% w/w of 10 nm diameter silica nanoparticles. The storage modulus (G′) and loss modulus (G″) of the oil was increased by the presence of nanoparticles. This increase was at least one order of magnitude over the entire observed temperature range for temperatures greater than 17.5 degrees Celsius.

FIG. 3. Comparison of shear viscosity as a function of temperature for canola oil based lubricants. The viscosity of the oil was found to increase with increasing weight percentage of 10 nm silica nanoparticles.

FIG. 4. A comparison of the mechanical viscoelastic properties for the pure canola oil and the canola oil with 10% w/w of 10 nm diameter silica nanoparticles. The storage modulus (G′) and loss modulus (G″) of the oil was increased by the presence of nanoparticles.

FIG. 5. Comparison of shear viscosity as a function of temperature for soybean oil-based lubricants. The viscosity of the oil was found to increase with increasing weight percentage of 15 nm diameter magnesium hydroxide nanoparticles.

FIG. 6. A comparison of the mechanical viscoelastic properties for the pure soybean oil and the soybean oil with 10% w/w of 15 nm diameter magnesium hydroxide nanoparticles. The storage modulus (G′) and loss modulus (G″) of the oil was increased by the presence of nanoparticles.

FIG. 7. Thermograms of pure canola oil and canola oil with 10% w/w of 10 nm diameter silica nanoparticles. These differential scanning calorimetry results indicate the transition from the liquid regime (high temperatures) to the gel-like regime (low temperatures) occurs at the pour point temperature of canola oil (˜−17° C.).

FIG. 8. A comparison of the mechanical viscoelastic properties for the pure soy wax and the soy wax with 10% w/w of 10 nm diameter silica nanoparticles. The storage modulus (G′) and the loss modulus (G″) of the oil in the liquid-regime was increased by more than one order of magnitude in the presence of nanoparticles.

FIG. 9. Thermograms of the pure soy wax and the soy wax with 10% w/w of 10 nm diameter silica nanoparticles. These differential scanning calorimetry results indicate the transition from the liquid regime (high temperatures) to the wax regime (low temperatures) occurs at the melting temperature of soy wax (˜50° C.).

FIG. 10. Schematic drawing of a triglyceride. A triglyceride can be divided into the polar head group and the three aliphatic chains attached to the head group.

FIG. 11. Change of shear viscosity as a function of temperature (a cooling rate of 1° C./min and shear stress of 50 pa) for (a) soybean oil, (b) corn oil and (c) canola oil. The open black squares are experimental data and the black line is the modified Andrade fit.

FIG. 12. Shear viscosity as a function of temperature (a cooling rate of 1° C./min and varied shear stresses of 50,100, and 200 Pa) for soybean oil. Increases in the shear stress result in an increase in the viscosity deviation temperature.

FIG. 13. Change of storage and loss modulus as a function of temperature (a cooling rate of 1° C./min and shear stress of 0.1 Pa, frequency of 1 Hz) for (a) soybean oil, (b) corn oil, and (c) canola oil.

FIG. 14. Loss angle versus frequency for soybean oil at −8° C.

FIG. 15. Shear viscosity as a function of temperature (a cooling rate of 1° C./min and shear stress of 100 Pa) for soybean oil with 5 wt. % of various hydrocarbon additives.

FIG. 16. Comparison of the change of storage and bulk modulus as a function of temperature (a cooling rate of 1° C./min, shear stress of 0.1 Pa, and a frequency of 1 Hz) for pure soybean oil and 5 wt. % 1-decene in soybean oil.

FIG. 17. Shear viscosity as a function of temperature (a cooling rate of 1° C./min and shear stress of 100 Pa) for soybean oil with 5 and 10 wt. % additives of 1-decene and n-decane.

FIG. 18. DSC cooling thermographs for soybean oil with hydrocarbon additives. The measurements were performed using a cooling rate of 1° C./min and the graphs were offset by 0.05 W/g in the y-axis for clarity.

FIG. 19. (a) Comparison of the shear viscosity as a function of temperature for 5 wt. % glycerol in soybean oil and pure soybean oil (a cooling rate of 1° C./min and shear stress of 100 Pa). (b) Modulus as a function of temperature for 5 wt. % glycerol in soybean oil (a cooling rate of 1° C./min, shear stress of 0.1 Pa, and a frequency of 1 Hz).

FIG. 20. Shear viscosity as a function of temperature (a cooling rate of 1° C./min and shear stress of 100 Pa) for soybean oil with 5 wt. % 1-decene and varying concentrations of glycerol.

FIG. 21. Shear viscosity dependence on the concentration of SiOₓ nanoparticles added to castor oils.

FIG. 22. Shear viscosity as a function of temperature (a cooling rate of 1° C./min and shear stress of 100 Pa) for soybean oil with varying concentrations of SiOₓ nanoparticles.

FIG. 23. The (a) storage and (b) loss modulus as a function of temperature (a cooling rate of 1° C./min, shear stress of 0.1 Pa, and a frequency of 1 Hz) for pure soybean oil and different concentrations of nanoparticles in soybean oil.

FIG. 24. Comparison of storage (G′) and loss modulus (G″) as a function of temperature (a cooling rate of 1° C./min, shear stress of 0.1 Pa, and a frequency of 1 Hz) for pure soybean oil and soybean oil with 10 wt. % 10 nm diameter SiOₓ nanoparticles.

FIG. 25. Shear viscosity versus shear rate for (a) pure soybean oil and (b) soybean oil with 10 wt. % 10 nm diameter SiOₓ nanoparticles.

Table 1. Comparison of the viscosity deviation temperature to the pour point temperature of several pure vegetable oil systems.

Table 2. Comparison of the temperature of the G′ and G″ crossover to the pour point temperature of several vegetable oil systems.

Table 3. Comparison of the physical properties of the hydrocarbons added to vegetable oil systems.

Table 4. Comparison of the viscosity deviation temperature to the pour point temperature of soybean oil systems with hydrocarbon additives

### DETAILED DESCRIPTION OF THE INVENTION

Referring to the drawings, like numerals indicate like elements and the same number appearing in more than one drawing refers to the same element. In addition, hereinafter, the following definitions apply:

The expression “vegetable oil derived material” refers to one or more natural vegetable oils or a synthetic product derived from one or more natural vegetable oils.

The term “nanoparticle” refers to particles having an average cross-sectional dimension (e.g., diameter, thickness etc.) generally less than about 1,000 nanometers. In some embodiments, compositions of the present invention comprise nanoparticles having a cross-section dimension selected over the range of about 1 nanometer to about 100 nanometers, and preferably for some applications selected over the range of about 10 nanometer to about 50 nanometers.

The term “average cross-sectional dimension” in the context of the present methods and compositions refers to the average cross sectional dimension of a nanoparticle. For spherical and substantially spherical (deviations from absolutely spherical geometry of less than 10%) nanoparticles, the cross sectional dimension refers to the average diameter of the nanoparticle. For nonspherical nanoparticles, the cross section dimensional refers to the average length of the largest dimension of the particle.

The term “vegetable oil” refers to oils derived from plant materials. Vegetable oils useful in the materials of the present invention may be hydrogenated or nonhydrogenated. Vegetable oils in the present methods and compositions include, but are not limited to, soy bean oil; sunflower oil, corn oil, canola oil, castor oil, cottonseed oil, peanut oil, olive oil, sunflower oil, rapeseed oil, and safflower oil. An example of a vegetable oil useful in the present compositions and methods is soybean oil. Pure soybean oils typically have between 10-35% Oleic fatty acid with about 60-90% of the fatty acids being unsaturated.

The term “wax” refers to material that is typically solid and is firm but not brittle. Waxes are generally malleable. Waxes typically have a melting point above approximately 45° C. Waxes of the present invention may comprise one or more triglyceride components. A wax useful in a specific embodiment of the present invention is a 100% soybean oil in the wax without additives, wherein the wax has a melting point of about 122 F(50 degrees Celsius).

The expression “synthetic wax derived from one or more vegetable oils” refers to waxes that are generated by synthetic pathways involving one or more vegetable oils as starting materials, including hydrogenation. A opposed to “true waxes”, such synthetic waxes are not naturally occurring, but rather are synthesized using natural materials, such as vegetable oils, as starting materials, precursors and/or additives. In some embodiments, the base waxes derived from vegetable oils are formed by the use of hydrogenation.

The expression “triglyceride-based wax” refers to a wax that comprises one or more triacylglycerol compounds. In some embodiments, a triglyceride-based wax of the present invention has a triglyceride component that is at least 20% by mass of the composition. Preferably for some applications the triglyceride components of a wax of the present invention comprises a triglyceride component having a concentration selected over the range of 20% to 80% by mass, and more preferably for some application comprises a triglyceride component having a concentration selected over the range of 20% to 50% by mass.

The terms “triglyceride” and “triacylglycerol” are used synonymously in the present description and refer to glyceride in which the glycerol is esterified with three fatty acids. Triglycerides are a main constituent of vegetable oil and animal fats. Some triglycerides have the formula:

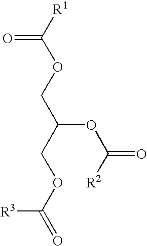

wherein R¹, R², and R³, are each independently substituted or unsubstituted aliphatic hydrocarbyl groups. Aliphatic hydrocarbon groups include alkyl groups and alkenyl groups have one or more double bonds. Substituted aliphatic hydrocarbyl groups include groups having one or more non hydrocarbon substituents, such as one or more hydroxyl groups, carbalkoxy group, alkoxy group, aldehyde group and/or alcohol group.

The term “hardness” refers to is the characteristic of a solid material expressing its resistance to permanent deformation. Hardness can be characterized by using the Brinell Hardness test method. This test can be implemented using a glass indenter of 16 mm diameter with an average 2 kg force applied. Hardness is calculated from the formulas associated with this test measurement and compared on the HB hardness scale.

The expression “mechanical stability” refers to the characteristic of the ability of a mechanical property such as G′ or G″ to remain constant for a given set of conditions over a broad range of temperatures.

The term “surfactant” refers to any chemical compound that reduces surface tension of a liquid when dissolved into it, or reduces interfacial tension between two liquids, or between a liquid and a solid.

As used herein, “nanosized” refers to features having at least one physical dimension (e.g. height, width, length, diameter etc.) ranging from a few nanometers to a micron, including in the range of tens of nanometers to hundreds of nanometers.

The term “viscosity” refers to a measure of a fluid's resistance to flow. It is often expressed in terms of the time required for a standard quantity of the fluid at a certain temperature to flow through an orifice of standard dimensions. The higher the value, the higher the viscosity. Viscosity is a variable which typically varies with temperature.

The present invention relates to the use of nanoparticles as additives to improve the physical characteristics of bio-based lubricants and waxes, such as lubricant and waxes derived from vegetable oils. Lubricants of the present invention comprising nanoparticle containing materials derived from vegetable oils exhibit enhanced lubrication properties at high and low temperatures, such as shear viscosities greater than similar vegetable oil-based materials not having a nanoparticle component. For example, upon incorporation of a nanoparticle phase, the shear viscosity of canola oil was doubled at ambient temperatures and increased an order of magnitude at very low temperatures as compared to pure canola oil. Waxes of the present invention comprising nanoparticle containing materials derived from vegetable oils exhibit enhanced mechanical properties, such as improved the rigidity, hardness and resistance of the wax. For example, the rigidity and resistance of soy wax was increased by an order of magnitude at high temperatures upon incorporation of a nanoparticle phase.

The effects of introducing nanoparticles into vegetable oils to form biodegradable lubricants and waxes is described. We show that the inclusion of nanoparticles in these systems significantly improves the physical properties critical for the desired applications of these materials.

### I. Vegetable Oil Based Lubricants

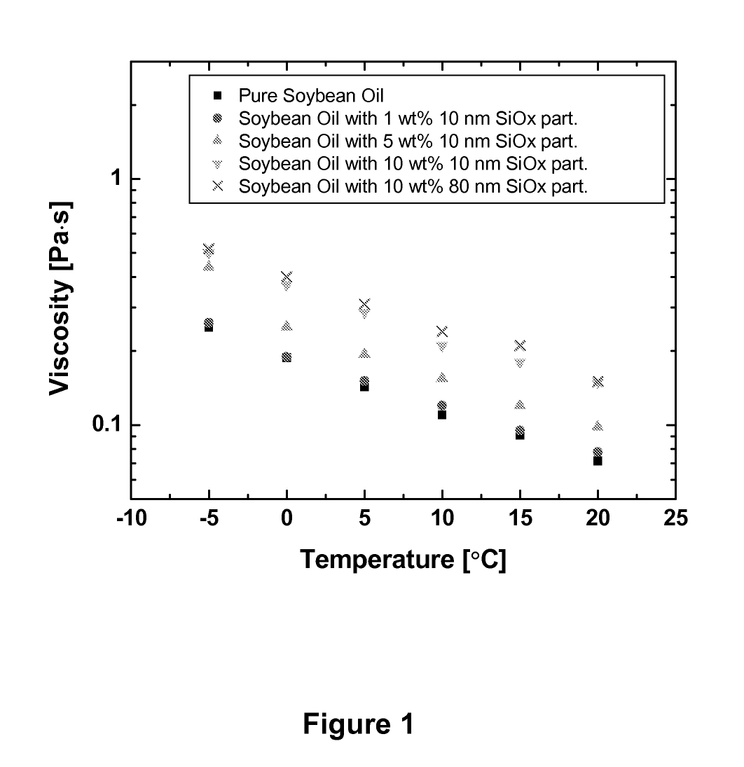

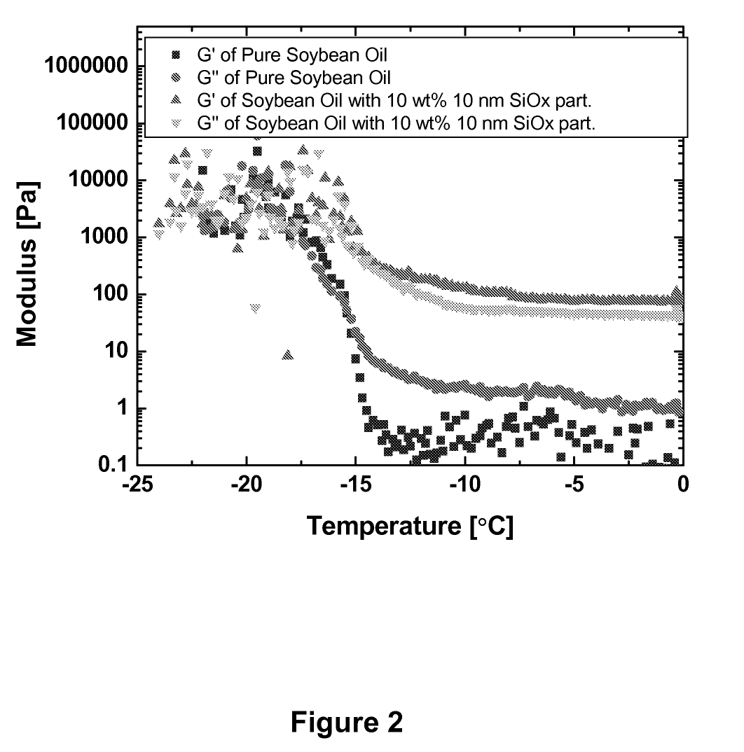

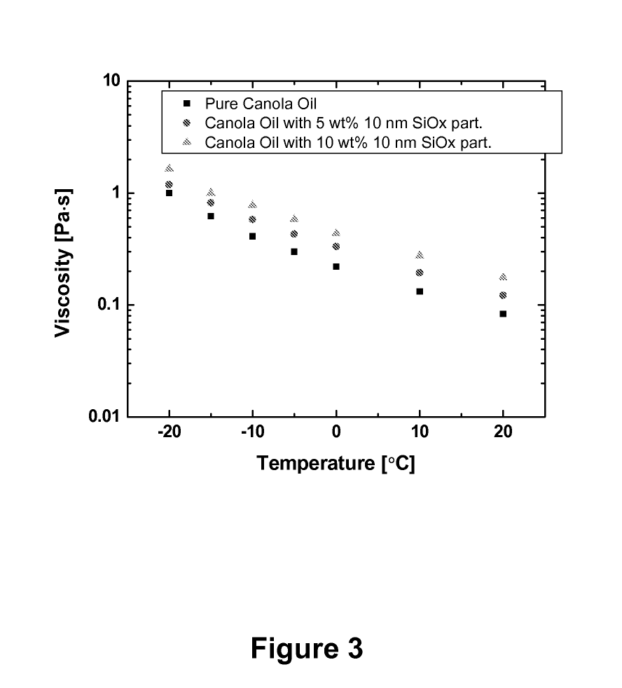

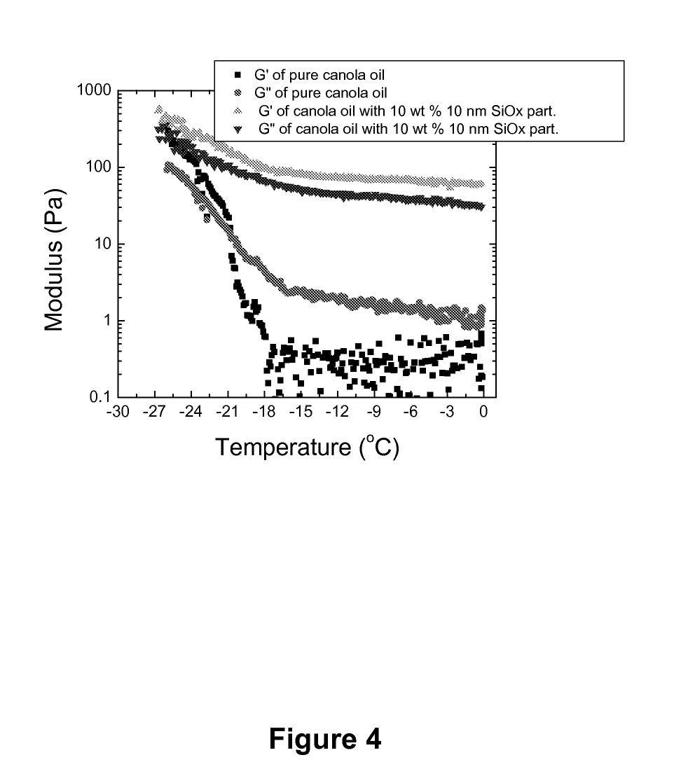

FIGS. 1-4 demonstrate that the introduction of silica nanoparticles into vegetable oil systems increases both the viscosity (FIGS. 1 and 3) and mechanical properties (FIGS. 2 and 4) of the oil. These physical properties are of great importance in producing commercially viable biodegradable vegetable oil based lubricants.

FIG. 1 provides a comparison of shear viscosity as a function of temperature for soybean oil based lubricants. The viscosity of the oil was found to increase with increasing weight percentage of 10 nm silica nanoparticles. The viscosity of the 10% w/w nanoparticle oil was more than double that of the pure soybean oil. The 10% w/w 80 nm silica nanoparticles also doubles the viscosity of the pure oil. Viscosity values were taken at a shear rate of 200 s⁻¹.

FIG. 2 provides a comparison of the mechanical viscoelastic properties for the pure soybean oil and the soybean oil with 10% w/w of 10 nm diameter silica nanoparticles. The storage modulus (G′) and loss modulus (G″) of the oil was increased by the presence of nanoparticles. This increase was at least one order of magnitude over the entire observed temperature range, particularly above about −15 degrees Celsius. Measurements were taken using a 1° C./minute shear rate at 1 Hz frequency and a 0.1 Pa shear stress.

FIG. 3 provides a comparison of shear viscosity as a function of temperature for canola oil based lubricants. The viscosity of the oil was found to increase with increasing weight percentage of 10 nm silica nanoparticles. The viscosity of the 10% w/w nanoparticle oil was about double that of the pure canola oil. Viscosity values were taken at a shear rate of 200 s⁻¹.

FIG. 4 provides a comparison of the mechanical viscoelastic properties for the pure canola oil and the canola oil with 10% w/w of 10 nm diameter silica nanoparticles. The storage modulus (G′) and loss modulus (G″) of the oil was increased by the presence of nanoparticles. This increase was at least one order of magnitude over the entire observed temperature range. Measurements were taken using a 1° C./minute shear rate at 1 Hz frequency and a 0.1 Pa shear stress.

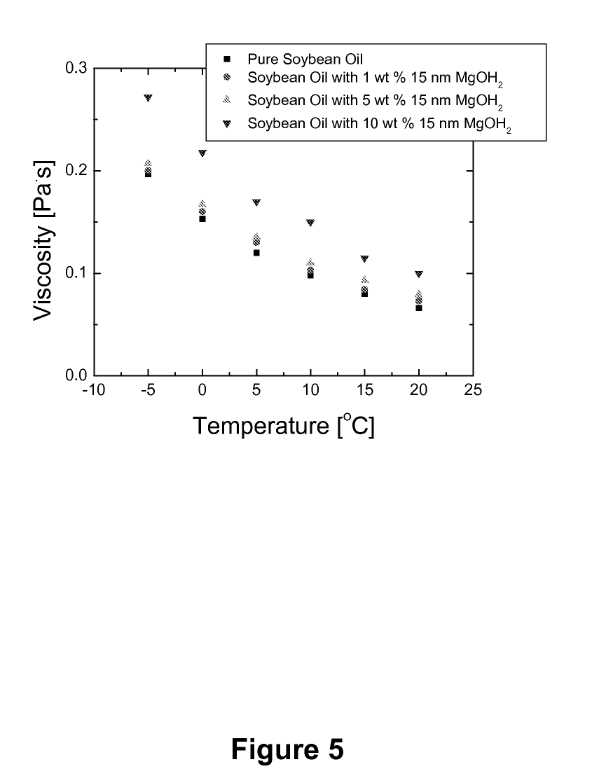

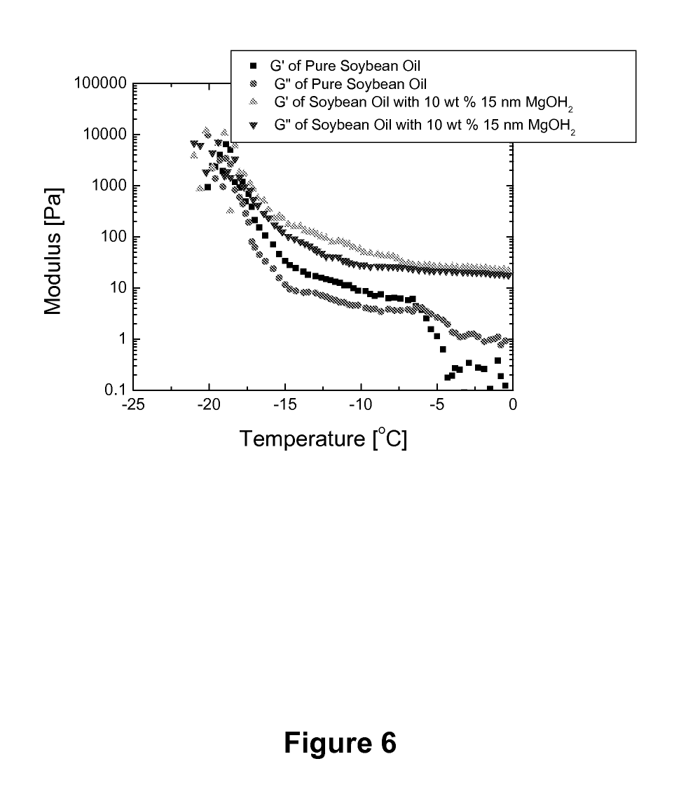

In FIGS. 5 and 6 provide experimental results showing the influence of the addition of 15 nm diameter Mg(OH)₂ particles to vegetable oil materials. In combination with the data provided in FIGS. 1-4, these results show that independent of the type of nanoparticles used, there is a clear improvement to the lubrication property of the vegetable oils.

FIG. 5 provides a comparison of shear viscosity as a function of temperature for soybean oil based lubricants. The viscosity of the oil was found to increase with increasing weight percentage of 15 nm diameter magnesium hydroxide nanoparticles. The viscosity of the 10% w/w nanoparticle oil was about 1.5 times that of the pure soybean oil. Viscosity values were taken at a shear rate of 200 s⁻¹.

FIG. 6 provides a comparison of the mechanical viscoelastic properties for the pure soybean oil and the soybean oil with 10% w/w of 15 nm diameter magnesium hydroxide nanoparticles. The storage modulus (G′) and loss modulus (G″) of the oil was increased by the presence of nanoparticles. This increase was at least one order of magnitude over the entire observed temperature range. Measurements were taken using a 1° C./minute shear rate at 1 Hz frequency and a 0.1 Pa shear stress.

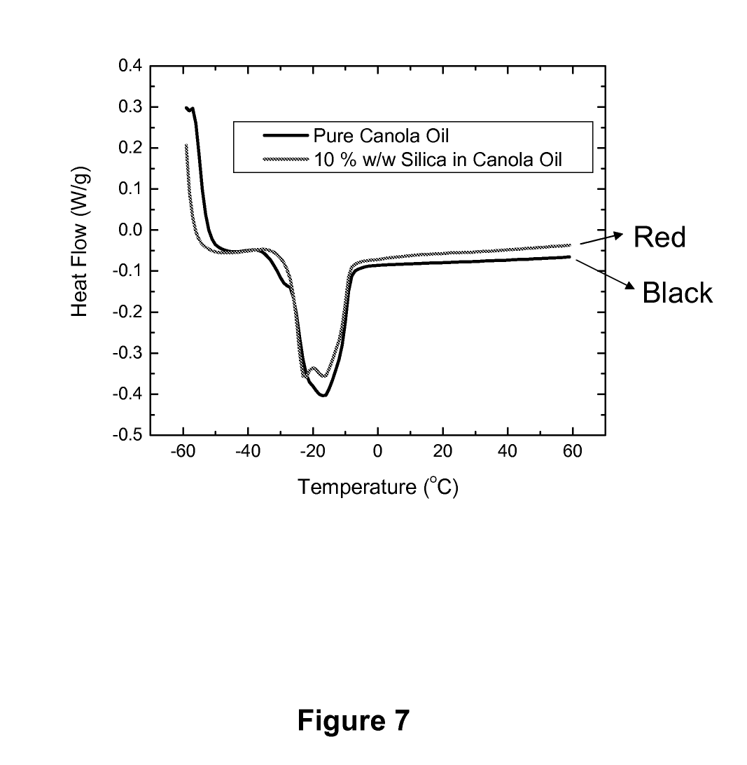

FIG. 7 provides thermograms of pure canola oil and canola oil with 10% w/w of 10 nm diameter silica nanoparticles. These differential scanning calorimetry results indicate the transition from the liquid regime (high temperatures) to the gel-like regime (low temperatures) occurs at the pour point temperature of canola oil (˜−17° C.). The presence of the nanoparticles was not found to influence the temperature at which this transition occurred.

### II. Vegetable Oil Based Waxes

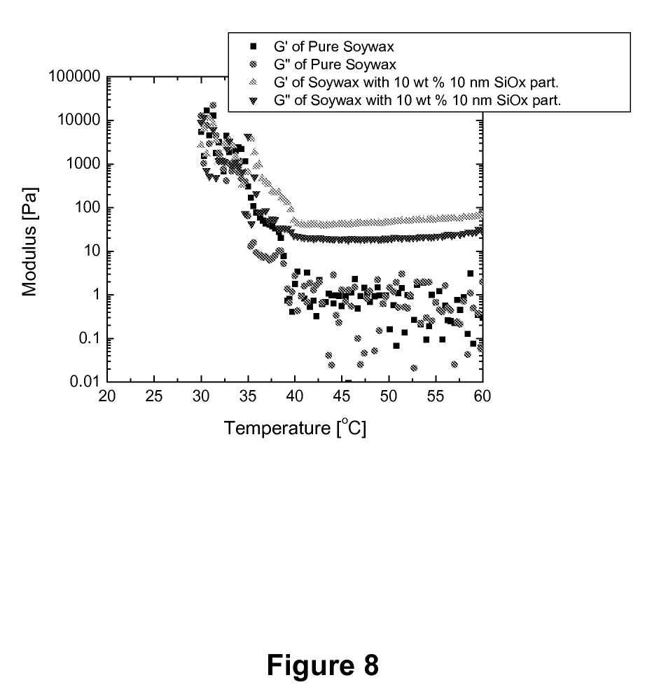

The introduction of nanoparticles into vegetable oil based waxes of the present invention enhances the mechanical properties (FIG. 8) of the wax. Experimental results indicate that a stronger, more rigid wax is achieved via the introduction of nanoparticles into vegetable oil based wax materials. The present wax compositions having a nanoparticle phase was further analyzed using the Brinell hardness test. The hardness values of pure wax was observed to be 0.90±0.17 Pa and wax with 10 wt. percent silica of 1.34±0.18 Pa which shows that the wax with the nanoparticles is harder.

FIG. 8 provides a comparison of the mechanical viscoelastic properties for the pure soy wax and the soy wax with 10% w/w of 10 nm diameter silica nanoparticles. The storage modulus (G′) and the loss modulus (G″) of the oil in the liquid-regime was increased by more than one order of magnitude in the presence of nanoparticles. A corresponding increase in the mechanical properties was not observed for the waxes at temperatures below the melting point, however, this difference is outside of the measurable limit of our instrument. To test the solid like regime of the wax we employed the Brinell hardness test. Measurements were taken using a 1° C./minute shear rate at 1 Hz frequency and a 0.1 Pa shear stress.

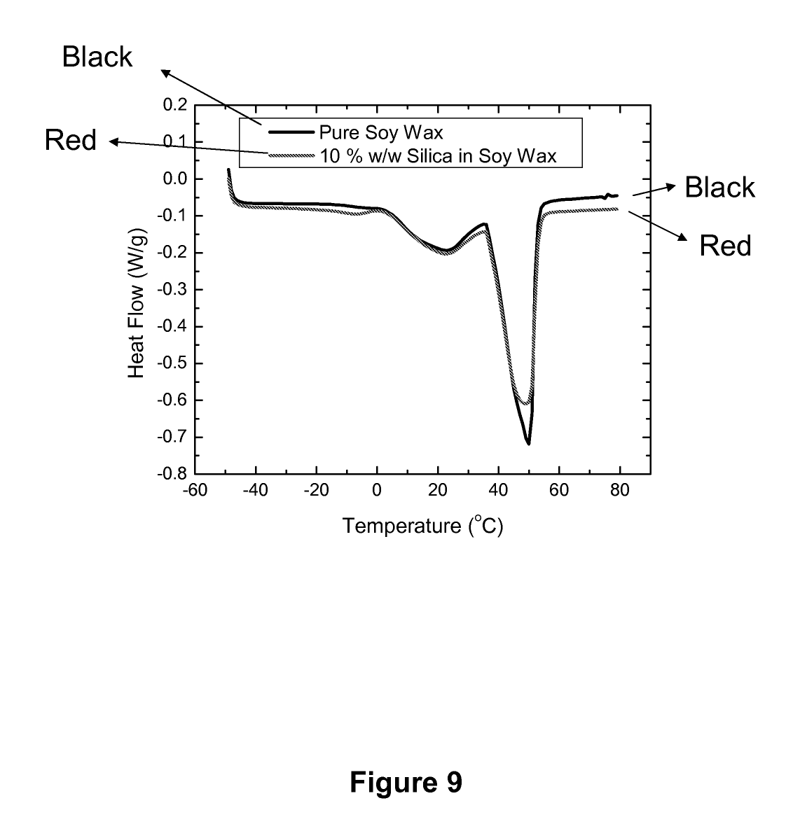

FIG. 9 provides thermograms of the pure soy wax and the soy wax with 10% w/w of 10 nm diameter silica nanoparticles. These differential scanning calorimetry results indicate the transition from the liquid regime (high temperatures) to the wax regime (low temperatures) occurs at the melting temperature of soy wax (˜50° C.). The presence of the nanoparticles was not found to influence the temperature at which this melting transition occurred.

The results provided here demonstrate that the introduction of nanoparticles increases the viscosity and mechanical properties of vegetable oil based lubricants and waxes. These results indicate that the physical properties of these vegetable oil based systems can be tailored to the desired specifications by varying the composition and type of added nanoparticles.

Applications of the compositions and methods of the present invention include, but are not limited to, lubricants (engines particularly 2-stroke engines), Transformer oil, Greases (moving parts, bearings, chains, engines, hydraulics), Waxes (coatings, car polish, seals, food preparation, pharmaceuticals, cosmetics, candles) and Cutting fluids.

In an embodiment, a nanoparticle-containing wax of the present invention is made via the following method. Nanoparticles are first thermally processed to remove any volatile materials, including water and hydrocarbons. In an embodiment, for example, the nanoparticles are heated to a temperature of approximately 140 degrees Celsius or greater under vacuum conditions for a period of at least 48 hours. The nanoparticles are subsequently cooled to room temperature by lowering the temperature under vacuum conditions. The wax component (e.g., one or more waxes derived from vegetable oils) is provided and heated to a temperature above its melting point to provide a phase change in the wax component from solid to liquid. In an embodiment, for example, the wax is heated using a liquid temperature bath to a temperature selected over the range of 60 degrees Celsius to 80 degrees Celsius. The nanoparticles are added to the wax component in the liquid phase and mixed so as to distribute the nanoparticles throughout the liquid phase. In some embodiments, for example, mixing of the mixture of nanoparticles and liquefied wax(es) is achieved via stirring for a period greater than or equal to 2 hours at a temperature above the melting point of the wax(es). The temperature is then lowered to cause a phase change in the wax component from liquid to solid, thereby generating the nanoparticle-containing wax of the present invention.

In an embodiment, a nanoparticle-containing lubricant of the present invention is made via the following method. Similar to the description above relating to waxes of the present invention, the nanoparticles are first thermally processed to remove any volatile materials, including water and hydrocarbons. In an embodiment, for example, the nanoparticles are heated to a temperature of approximately 140 degrees Celsius or greater under vacuum conditions for a period of at least 48 hours. The nanoparticles are subsequently cooled to room temperature by lowering the temperature under vacuum conditions. The vegetable oil component (e.g., one or more vegetable oils) is provided in the liquid phase at room temperature. The nanoparticles are added to the vegetable oil component in the liquid phase and mixed so as to distribute the nanoparticles throughout the liquid phase. In some embodiments, for example, mixing is achieved via stirring the mixture of nanoparticles and vegetable oil(s) at room temperature for a period greater than or equal to 24 hours, thereby generating the nanoparticle-containing lubricant of the present invention.

### EXAMPLE 1

### Rheological Characterization of the Pour Point Temperature for Pure and Additive Enhanced Vegetable Oil-Based Lubricants

### Abstract

Rheological measurements, including viscosity sweeps and small-amplitude oscillatory shearing, was used to characterize the pour point and low temperature behavior of vegetable oil-based lubricants. The shear viscosity of the oils at temperatures above the pour point followed a modified Andrade equation, however, at temperatures below the pour point the measured viscosity of the oils deviated from the fit. Oscillatory measurements of the storage (G′) and loss (G″) moduli also indicated a transition behavior at the pour point temperature of the oils. Vegetable oils at temperatures above the pour point had G′<G″ indicating that the oil was liquid-like, but at temperatures below the pour point had G′>G″ indicating that the oil was gel- or solid-like in nature. This type of crossover in G′ and G″ is often associated with sol-gel systems undergoing a gelation process. Gelation of the vegetable oil systems was further demonstrated by the frequency independence of the loss angle close to the pour point temperature. In addition, the influence of additives on the low temperature properties of the vegetable oils was characterized using the same rheological methodologies. Both organic straight chain hydrocarbons, ranging from hexane to eicosane, and inorganic silicon oxide nanoparticles were characterized as potential additives to pure vegetable oils. A blend of 1-decene and glycerol additives was found to be the most beneficial in creating a vegetable oil lubricant by depressing the pour point temperature (by ˜6° C.) and raising the oil viscosity (by more than a factor of 2).

### Introduction

Vegetable oils are a biodegradable, naturally occurring, and renewable resource that may in the near future replace petroleum-based products as lubricants for a wide range of applications [1-9]. Current limitations to the widespread use of vegetable oil lubricants primarily arise from their poor low temperature properties. The low temperature property of greatest significance is the pour point which characterizes the temperature at which the lubricant ceases to flow under the influence of gravity. Pour point temperatures are determined for all oils using a standardized ASTM test and equipment [10] that are difficult to replicate and that cannot easily be interpreted in terms of conventional thermophysical properties or measurement techniques.

Pure vegetable oils have pour points that are well above those of optimized petroleum-based products. For example, the vegetable oils of interest to this work, e.g. soybean, canola and corn oils, exhibit pour points in the neighborhood of −18° C. In contrast, pure petroleum-based oils have pour points ranging from 49° C. to −18° C. or lower, but commercial oil products when optimized with specially designed additives exhibit pour point temperatures less than −40° C. [11-12]. The most common pour point depressants in petroleum-based systems include poly(methyl methacrylate), polyacrylamides, Friedel-Crafts condensation products of chlorinated paraffin wax with naphthalene and phenol (often referred to as alkylaromatic polymers), and ethylene propylene olefins [12-13].

Pour point depressants similar to those used in petroleum-based lubricants are also of interest in vegetable oil systems. Due to the proprietary nature of many of these additives, the details and molecular-level understanding of these systems remains limited. Typical additives studied as pour point depressants in biodegradable lubricants include synthetic diesters and polyol esters, poly alpha olefins, polymethacrylate backbone branched polymers, and oleates [14]. Asaduaskas et al. [14] studied a number of pour point reducers and found that the addition of poly alpha olefins, mixtures of dimers and trimers of 1-decene, lead to the greatest reduction in the pour point temperatures of sunflower and soybean oils by 9° C. and 12° C., respectively. This work also noted that the addition of 0.4 wt. % 8000 amu poly(alkyl methacrylate) decreased the pour point temperatures of soybean oil and canola oil by 9° C. and 15° C., respectively [14]. Ming et al. [15] demonstrated that the addition of dihydroxy fatty acids reduced the pour point of palm oil-based systems by 7° C. These prior studies, however, have not characterized the effect of these additives on rheological properties. In crude oil-based systems the addition of pour point additives has been found to decrease the shear viscosity of the oil [16,17]. A comparable lowering of the viscosity of vegetable oil-based systems by the additives could potentially limit the applications for which they could function.

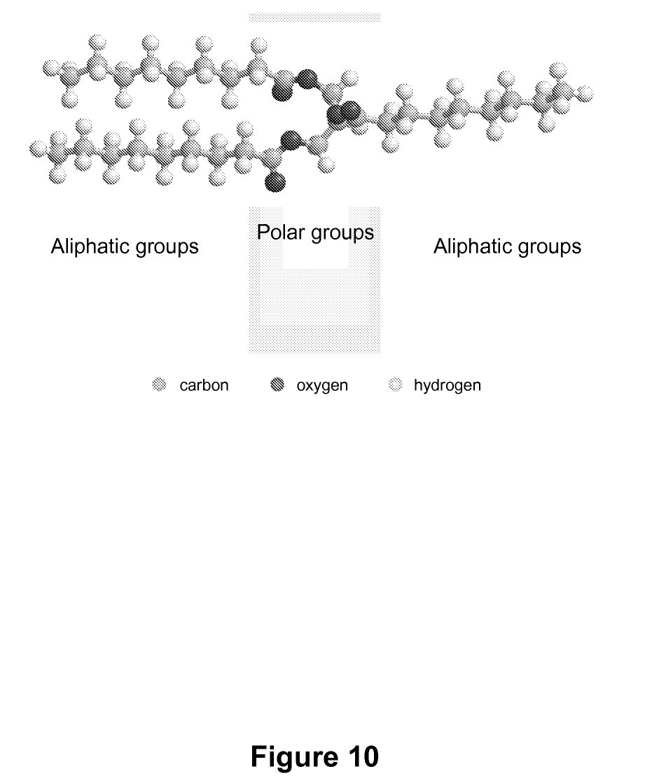

Here we begin by demonstrating that rheological characterization can determine the pour point of vegetable oils and that this temperature is associated with the gel formation of the oil. Next we consider the modification of the thermophysical properties of vegetable oils through the blending of pure oils with additives. We extend our current rheological study of vegetable oils to characterize the influence of various classes of additives, including both small molecule organics and inorganic nanoparticles, on the pour point behavior. Consequently, we have first considered additives that have chemical features similar to the chemical features of triglycerides, the main component of vegetable oils (see FIG. 10). Inorganic nanoparticles, which are commonly used as rheological modifiers in inks and paints, have also been studied as viscosity enhancers in vegetable oil-based systems.[18]

### Experimental Section

### Materials and Methods.

Chemicals. All of the vegetable oils used in the study were obtained from the local market. The hydrocarbon additives used in this study include n-eicosane purchased from Alfa Aesar, 94% pure 1-decene purchased from Aldrich, 99% pure n-hexadecane and 99% pure decane purchased from Acros Chemicals, and 99+% pure n-octane and 98.5% hexane reagents purchased from Sigma. The 99.5% pure glycerol used was purchased from Sigma. All nanoparticles used in this study were purchased from Nanostructured & Amorphous Materials Inc. The 99.5% pure 10 nm silicon oxide (SiOₓ) had a reported specific surface area of 640 m²/g and the 99% pure 80 nm silicon oxide had a reported specific surface area of 440 m²/g. Both SiOₓ samples had similar reported bulk densities of 0.063 g/cm³ and true densities of 0.063-0.068 g/cm³.

Sample Preparation The SiOₓ nanoparticles were placed in a vacuum oven at 140° C. for at least 48 hours in order to remove residual water and then immediately mixed with the vegetable oils. All blended systems were stirred for at least 24 hours prior to characterization.

Rheological Measurements. The rheological characterization was performed on a Bohlin CVO 50 constant stress rheometer (Malvern Instruments Ltd., Worcestershire, UK) equipped in a parallel plate geometry. The temperature was controlled by a thermal bath from 20° C. to below −25° C. at constant cooling rates of 0.5 or 1° C./min. To eliminate the effect of ice formation at low temperatures, the samples were held under a nitrogen purge.

Differential Scanning Calorimetry. The DSC thermograms were obtained using a TA Instruments Q100 DSC (New Castle, Del.). The dehydrated samples were scanned from 60° C. to −60° C. (or the desired lower temperature) at 1, 5, or 10° C./min. Duplicate samples were measured and at least two scans were performed for each sample. All measurements were made using sealed aluminum hermetic pans with at least 8 mg of sample, and an empty pan was used as a reference. Data was analyzed using Universal Analysis.

Pour Point Measurements The pour point temperatures of the vegetable oil systems were measured following the specifications of ASTM D97 [10]. A pour point apparatus was constructed in house based on the requirements of the ASTM test. The samples were heated to 50° C. for half an hour prior to the pour point measurements in order to remove any thermal history of the sample.

### Results and Discussion

### Rheological Characterization of the Pour Point

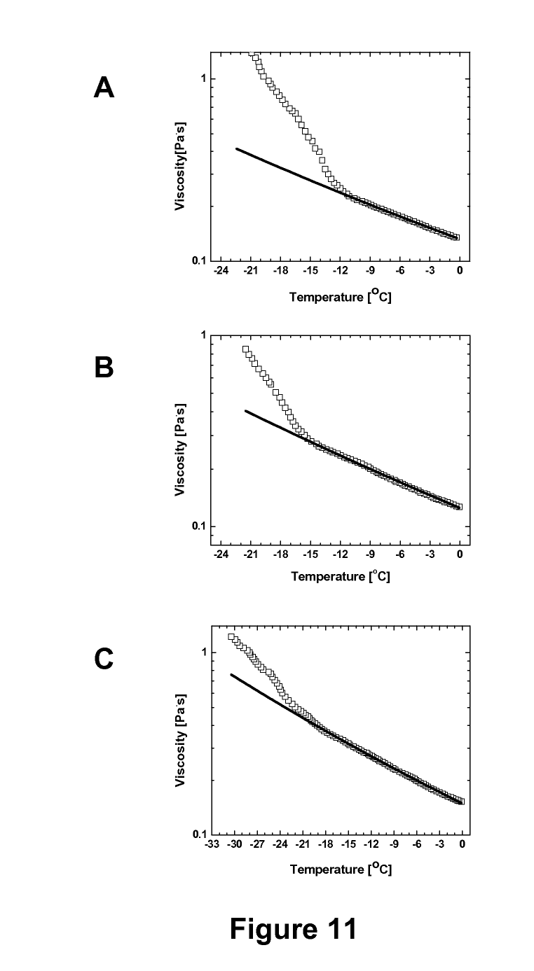

Rheological characterization to determine the pour point temperature of vegetable oil-based systems was first demonstrated by measuring the shear viscosity versus temperature during controlled cooling sweeps from temperatures above the pour point to temperatures below the pour point (see FIG. 11). The viscosity curves of all of the vegetable oils follow an exponential relationship at temperatures above the pour point and then deviate near the pour point temperature of the oil. Previous work by Abramovic and Klofutar has shown that the liquid viscosities of vegetable oil-based systems follow the empirical modified Andrade equation [19]:

ln η=A+B/T+C/T²

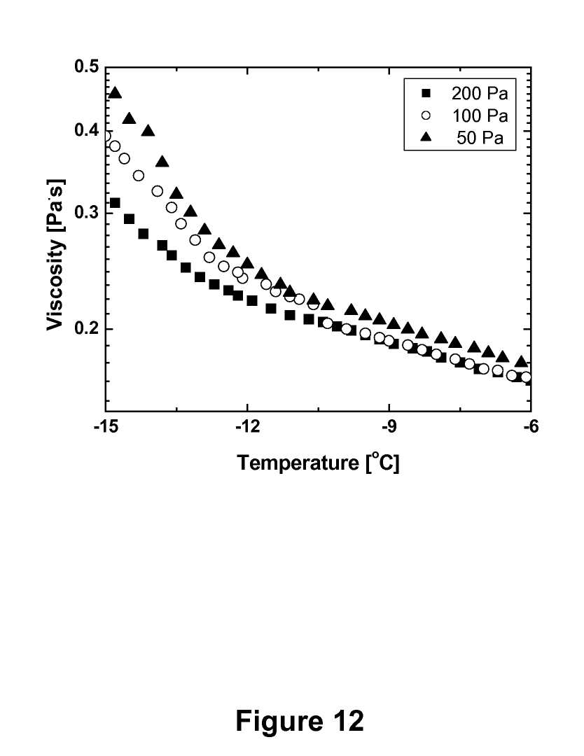

In all of these tests, the shear viscosity of the fluid follows a modified Andrade fitting at temperatures above the pour point indicating that the oil is in a liquid-like state. As the temperature of the oils is decreased to the pour point, however, the viscosity behavior of the oils begins to deviate from this functional form. As can be seen in FIG. 12 for soybean oil and is summarized in Table 1 for other oils, this deviation temperature is close to the pour point temperatures of the vegetable oil. The deviation in the viscosity curve has been characterized as a function of both shear stress and cooling rate since the pour point of petroleum-based systems arise as a result of increases in the yield stress of the system and has been shown to be dependent on the thermal history of the system [20-26]. The calculated pour point temperature decreases by ˜1-2° C. with increasing shear stress, but is nevertheless very near the pour point temperature range as measured in this work using the standard pour point characterization protocol described in ASTM D97. The decrease in pour point temperatures observed with increasing shear stresses suggests that higher stresses applied to the oil systems slow the mechanism necessary for the molecules to organize and the oil to gel. At slower cooling rates the deviation temperature tends to increase slightly and the viscosity of the system below the deviation temperature is greater than in systems characterized at faster cooling rates. This result suggests that slower cooling rates allow for more molecular aggregation and structural arrangement of the triglycerides, and therefore stronger gels are formed at higher temperatures.

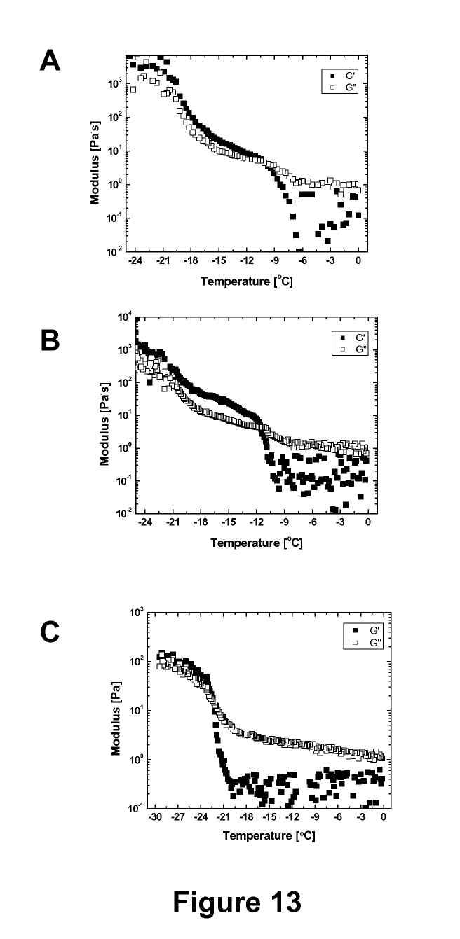

Small-amplitude oscillatory shear measurements also have been used to characterize the pour point transitions of vegetable oils. Such measurements using temperature sweeps at a constant cooling rate were performed within the viscoelastic regime at a frequency of ω=1 Hz and a shear stress of 0.1 Pa. For each of the pure soybean, canola, and corn oil samples in FIG. 13, at temperatures much greater than the pour point the storage modulus (G′) is smaller than the loss modulus (G″) indicating that the oil is more viscous and liquid-like (G′<G″). The storage modulus increases with decreasing temperature until it eventually crosses over and becomes larger than the loss modulus indicating that the oil in this regime is more elastic and solid-like (G′>G″). At temperatures below the crossover temperature, the storage modulus continued to increase and then leveled off at a value several orders of magnitude higher than the storage modulus at high temperatures. The temperature at the G′ and G″ crossover point and transition corresponds with the pour point temperature of the oil system. The temperature of the crossover point also was found to be independent of oscillation frequency. The observed behavior of the dynamic moduli as a function of temperature is similar to that extensively characterized for systems undergoing a sol to gel transition [27,28]. It is suggested that as the temperature of the vegetable oil systems are reduced from above to below the pour point temperature the structuring of the triglycerides begins to occur and a stronger gel is formed.

In a manner similar to that discussed previously for the shear viscosity measurements, at slow cooling rates the G′ and G″ crossover point occurs at a slightly lower temperature and leads to slightly higher moduli values. Again this result indicates that the slower cooling rates, and the resultant longer times near and at the pour point, enable a stronger gel to form due to molecular-level aggregation and structural arrangement. Gelation processes such as the pour point are time and temperature dependent, therefore, rheological characterizations should be performed at a rate similar to that defined in ASTM D97 in order to determine transition points that match the conventional pour point temperature. Analogous results have been observed in petroleum-based oils (see Table 2). Veneckatesan et al. [20] and Lopes de Silva et al. [21] have shown that the gelation point can be detected for crude oils using oscillatory measurements and that the determined gelation temperature is higher than the measured pour point temperature. Both of these studies, however, used slow cooling rates of 0.1 and 0.2° C./min [21], whereas Visinitin et al. [22] showed that the gelation temperature was similar to the pour point temperature at a faster cooling rate of 1° C./min and higher than the pour point for a slower cooling rate of 0.05° C./min.

The crossover of the G′ and G″ dynamic moduli is often considered a satisfactory characterization of gel behavior and the gelation temperature. This crossover point, however, is not a universal property of the gel point but has long been seen as an acceptable criterion for the characterization of the sol-gel transition [22, 27]. A more reliable and suitable rheological approach to determining gelation has been proposed by Chambon and Winter [28-31] and involves characterizing the frequency independence of the loss tangent. Under this criterion, the storage and loss moduli of the critical gel exhibit a power law scaling with frequency and at the gel point the loss angle is independent of the frequency such that

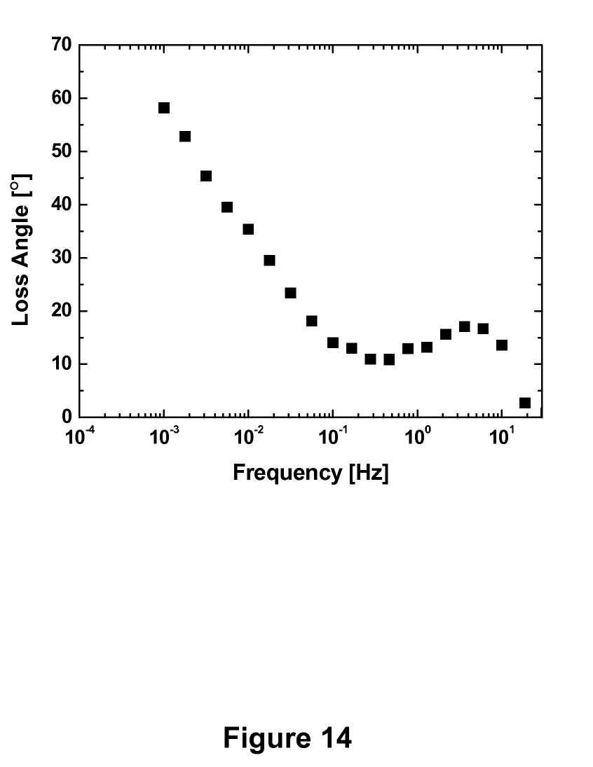

tan δ=tan(nπ/2)

where n is the relaxation exponent with 0<n<1. A gel is viscous if n is close to 1 and elastic if n is close to 0. Here we find that the loss angle for the vegetable oil-based systems behaves very similarly to what has been observed in branched polymers by Garcia-Franco et al. [27]. As shown in FIG. 14 the loss angle at low frequencies (w<0.1 Hz) decreases with increasing frequency and then reaches a plateau value which is independent of frequency (0.1<ω<1 Hz). Oscillatory measurements performed at higher frequencies (w>10 Hz) display further decreases in the loss angle to δ˜0°. The soybean oil system characterized in FIG. 14 was equilibrated at the desired temperature to ensure that the gel transition occurred and that the modulus was constant throughout the measurement. For example, the soybean oil examined at −8° C. was found to have a relaxation exponent of 0.25±0.01 indicating that the oil equilibrated at this temperature is solid-like in nature.

### The Role of Additives on the Pour Point

Vegetable oil-based lubricants have several physical properties that limit their widespread use, namely poor low temperatures properties as quantified by high pour point temperatures and low viscosity values relative to petroleum-based lubricants. Understanding the rheological impact of adding pour point depressants to the vegetable oil-based systems, as well as the effect of molecular structure and chemical functionality on depressing the pour point temperature, will be very useful for the design of vegetable oil-based lubricants. As previously discussed, many studies that have considered the use of pour point depressants in vegetable oil-based lubricants have used pour point depressants that are effective in petroleum products. The chemical nature of petroleum oils, which are mixtures of hydrocarbons, and vegetable oils are very different. Vegetable oils are essentially mixtures of triglycerides, which can be described as having a glycerol head group to which three aliphatic chains are attached (see FIG. 10). Therefore, it would be beneficial to determine which pour point depressants can operate in the most effective manner for the specific molecular architecture of vegetable oils.

### Aliphatic Additives

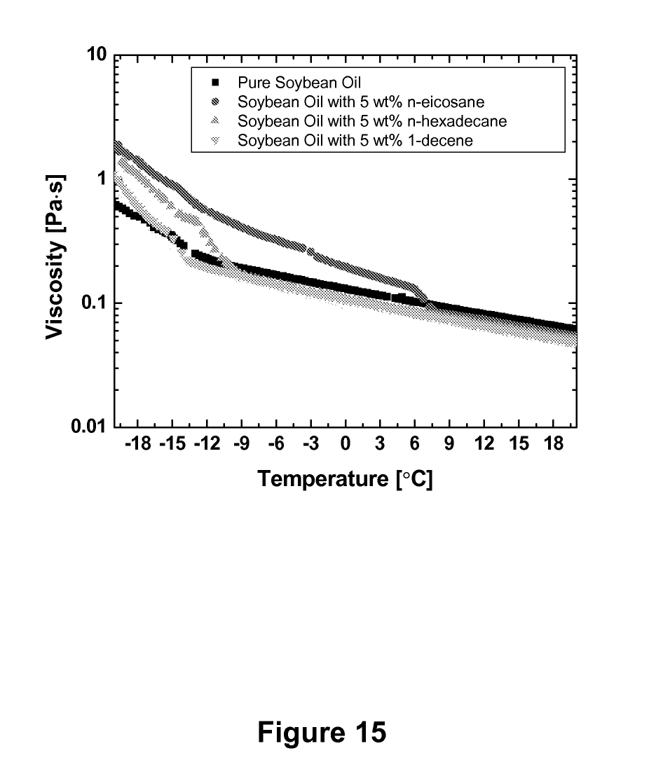

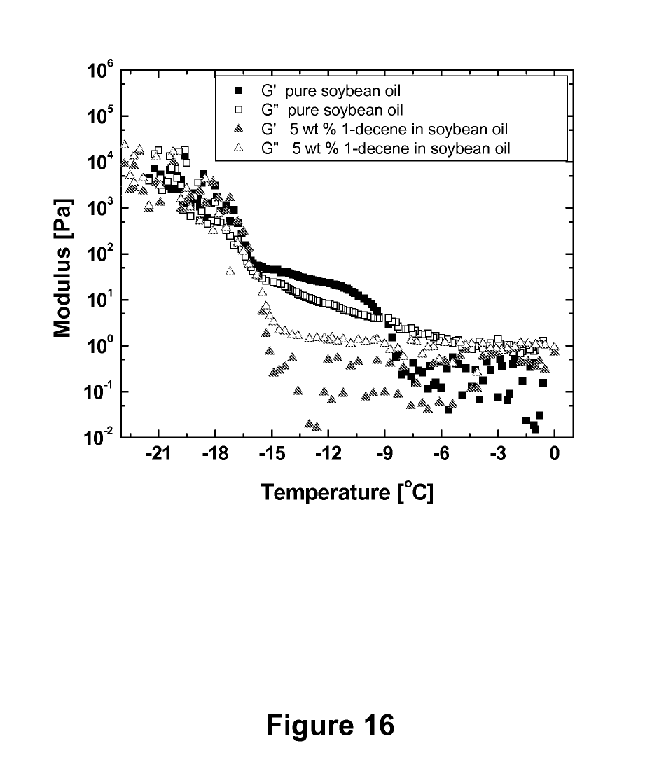

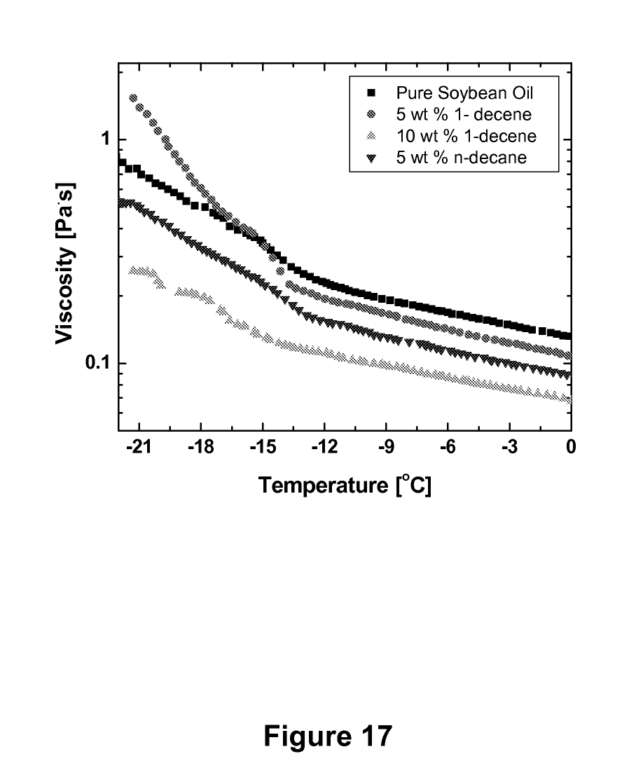

Aliphatic, straight chain hydrocarbons ranging from 6 to 16 carbons in length (see Table 3) have been characterized as additives in vegetable oils due to their chemical similarity to the tails of triglycerides. These hydrocarbons were first considered as additives to soybean oil in concentrations of 5 wt. %. FIG. 15 demonstrates the change in shear viscosity as the oil samples are cooled from above to below the pour point temperature at a constant rate of 1° C./min. As was previously shown for pure vegetable oils, the shear viscosity of the blended oil plus additive systems deviated from the empirical Andrade fit at temperatures at the pour point of the oils. A comparison of the fittings of the shear viscosity and the pour point temperatures of the mixture systems was performed and the results are presented in Table 4. Oscillatory rheology was also performed and the dynamic shear moduli again display a crossing of G′ and G″ at temperatures at the pour points of the oil systems (see FIG. 16). The inclusion of the hydrocarbon additives alters the observed pour point temperature from that of pure soybean oil and at temperatures above the pour point decreases the shear viscosity of the blend systems. The relative impact of the hydrocarbon additive is dependent on both the size and melting point of the additive. For longer hydrocarbon chains with higher melting points, such as eicosane and n-hexadecane, the pour point of the blended oil was higher by 15° C. and 3° C., respectively, than the pure soybean oil and at temperatures above the pour point the decrease in viscosity was roughly 10 to 18% from that of the pure oil. Smaller hydrocarbon additives with lower melting points, such as 1-decene, decane, octane, and hexanes, reduced the pour point temperature of the soybean oil systems while the viscosity of the blended oils was decreased by as much as ˜25%. At temperatures below the pour point all of the blend systems with hydrocarbons displayed substantially greater increases in viscosity with decreasing temperature than the pure soybean oil. The effect of additive size was compared to that of additive melting point by considering both decane and 1-decene hydrocarbons. Both of these additives have the same number of carbons and therefore nearly the same molecular size, but the presence of the double bond in 1-decene decreases its melting point to −63° C. from that of n-decane at −30° C. FIG. 17 shows that the pour point of the soybean oil blended with 5 wt. % 1-decene occurs at a lower temperature than for the soybean oil blended with 5 wt. % decane. It can therefore be concluded that the melting point of the additive plays a significant role in the pour point of the oil system while the additive size has little influence. Furthermore, the viscosity of the 5 wt. % decane system is lower than that of the 1-decene system. The presented pour point temperature and viscosity results indicate that 1-decene is the most promising of the hydrocarbons considered here for use as a pour point depressant. We have also considered the effect of adding 10 wt. % 1-decene to soybean oil and found that the viscosity of the oil decreases by a factor of 2.

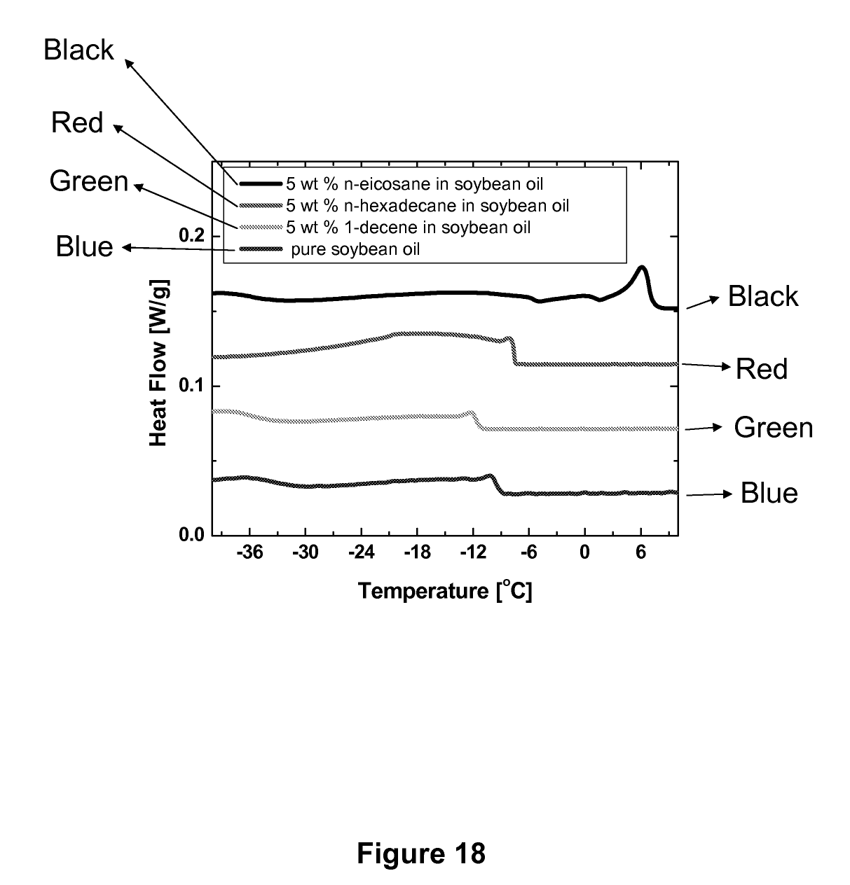

The effect of hydrocarbon additives on the pour point temperature of oils is further illustrated in the DSC thermographs of FIG. 18. The first peak of the scan occurs at the temperature at which the blend viscosity begins to deviate from the modified Andrade fit and corresponds to the onset of the pour point. The temperature of the first peak is observed to shift as a function of the hydrocarbon additive from 6.1° C. for n-eicosane to −12.6° C. for 1-decene. These results may explain the role the aliphatic additives play in affecting the pour point of the system. We believe that the hydrocarbon additives with high melting temperatures organize and in some cases crystallize causing the co-crystallization of the oil with the hydrocarbon. For the shorter hydrocarbons like 1-decene, the hydrocarbon can reduce the structure of the system and diminish the propensity for gelation to occur thereby allowing for lower pour points to be obtained.

### Glycerol Additive

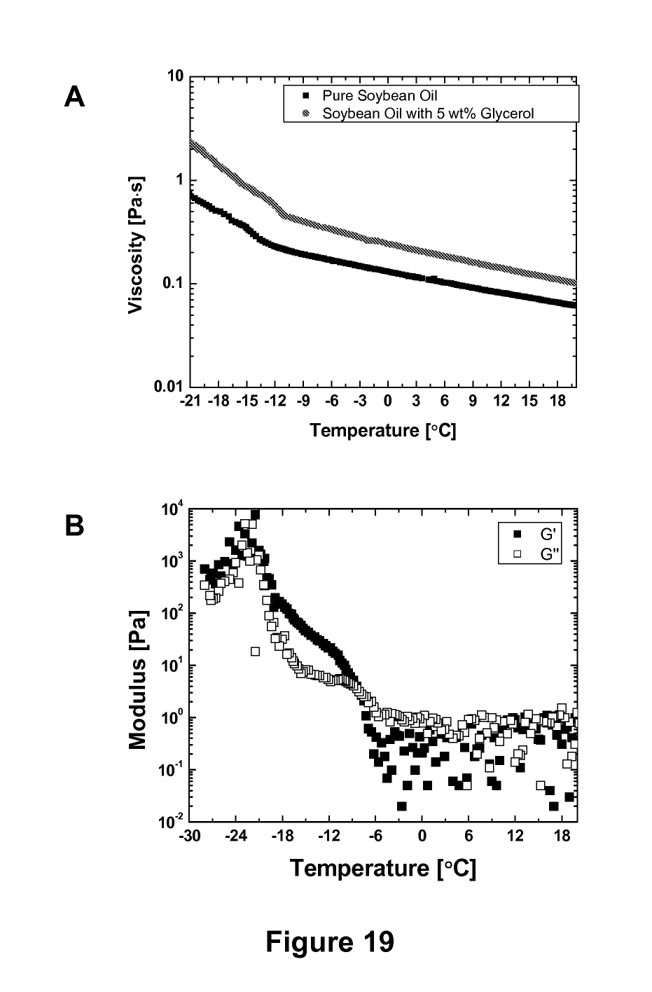

Glycerol, which has a chemical structure identical to the triglyceride headgroup, was added to the soybean oil in order to further understand the role of additives in the vegetable oil systems. The addition of glycerol increased the viscosity of pure soybean oil above the pour point temperature by approximately 30% to 50%, while the viscosity below the pour point temperature was more than twice that of the pure oil. The presence of the glycerol also increased the mechanical properties of the oil below the pour point temperature of the system by roughly doubling the G′ values and increasing the G″ values by an order of magnitude (see FIG. 19). These rheological characterization techniques have not detected, however, any shift in the pour point temperature in response to the glycerol additive. Consequently, glycerol can be considered as a vegetable oil additive for improving physical properties such as viscosity but should not be applied as a pour point depressant.

### Mixtures of Hydrocarbon Additives

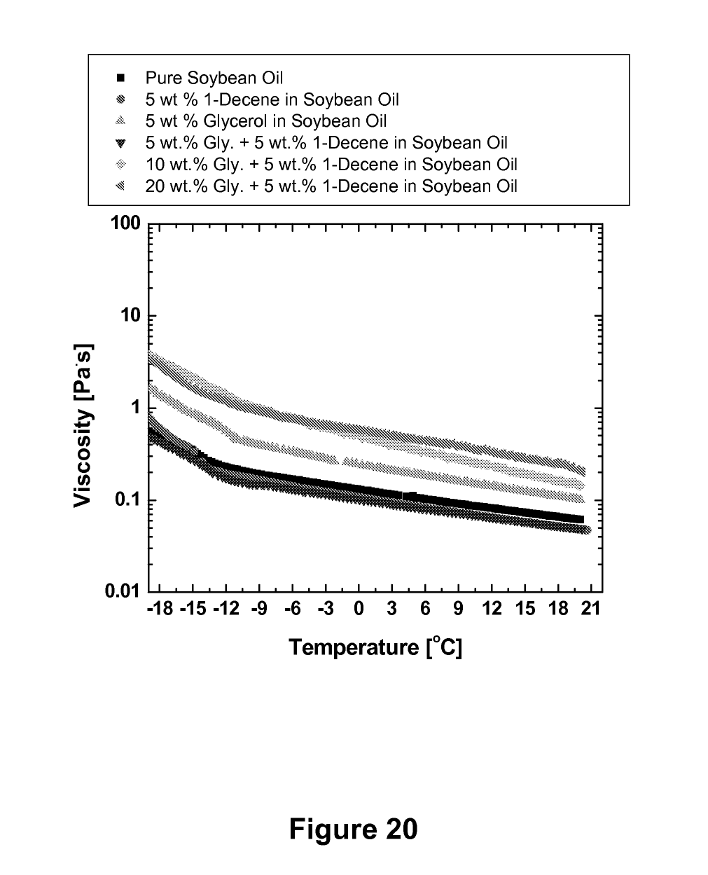

The inclusion of multiple additives in vegetable oils has been considered as a route to imparting the oil with the favorable attributes of the independent additives. As demonstrated in the preceding sections, the addition of 1-decene tended to lower both the pour point temperature and the viscosity of soybean oil, while the addition of glycerol was observed to thicken the oil systems but did not change the pour point temperature. An ideal oil system would have the lower pour point temperature accessible with the 1-decene and the higher viscosity of the glycerol additive. Therefore, the behavior of additive mixtures of 5 wt. % 1-decene and varying concentrations of glycerol were experimentally studied in soybean oil. The viscosity of the system increased as the glycerol concentration was raised from 5 to 20 wt. % as shown in FIG. 20. The viscosity of the mixture of soybean oil with 5 wt. % glycerol and 5 wt. % 1-decene was only slightly higher than the soybean oil with 5 wt. % 1-decene but was still lower than the pure soybean oil. On the other hand, the viscosity of the mixture of soybean oil with 10 wt. % glycerol and 5 wt. % 1-decene was more than double that of pure soybean oil. Both of these mixtures had the same pour point temperature of −15° C. as the soybean oil with 5 wt. % 1-decene additive. Mixtures with higher concentrations of glycerol were able to further increase the viscosity of the oil, e.g., the mixture of soybean oil with 20 wt. % glycerol and 5 wt. % 1-decene had a viscosity that was more than triple that of pure soybean oil. The addition of this amount of glycerol, however, appears to have impeded the effect of the 1-decene as a pour point depressant. The pour point temperature of the 20 wt. % glycerol and 5 wt. % 1-decene system was measured to be identical to the pure soybean oil at −9° C. These results clearly demonstrate that the pour point and viscosity of vegetable oils can be precisely tuned through the proper choice of the additives and their relative concentrations.

### Nanoparticle Additives

Nanoparticle additives in vegetable oil systems have been studied as a promising approach for modifying the thermal, rheological, and mechanical behavior of the oil and for creating lubricants with suitable properties for widespread application. In the literature the primary focus on nanoparticle additives in lubricating systems has been as tribo-active additives, containing tribologically active elements (P, S, Cl, Zn, N) used to reduce wear, as well as, anticorrosion additives created from alkaline earth metal hydroxides [18, 32-38]. Nanoparticles have long been used to modify the physical properties of polymeric based systems. For example, small amounts of organically-modified layered silicates, or nanoclays, have been used as rheological modifiers in paints and inks. Such types of nanoparticles have also been added to lubricating oils as thickening agents to create non-melting greases for high temperature applications [18]. Numerous studies have shown that the thermal, e.g. the glass transition temperature, and physical, e.g. mechanical, properties of polymer nanocomposite materials can be controlled by the amount and type of nanoparticle used [39-43].

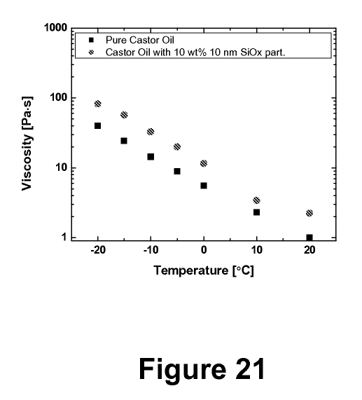

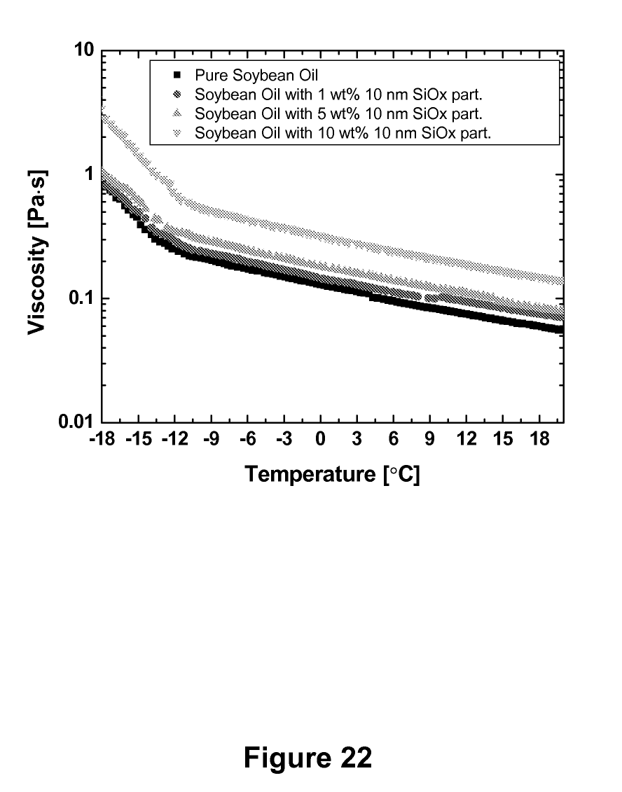

In this work silicon oxide (SiOₓ) nanoparticles were added in varying amounts to soybean, canola, and castor oil. The shear viscosity was first characterized as shown in FIGS. 1, 3 and 21 by holding the samples at constant temperatures. A concentration of 10 wt. % nanoparticles with an average diameter of 10 nm was shown to double the shear viscosity for all of the oils examined (see FIGS. 1, 3 and 21). The effects of the nanoparticle size were also considered in soybean oil. The addition of 80 nm SiOₓ particles in a 10 wt. % concentration, unlike the 10 nm diameter particles, led to a viscosity only slightly greater than that of the pure soybean oil (See FIG. 1). For soybean oil, the shear viscosity of the oil was also studied as it was cooled at a rate of 1° C./min (see FIG. 22). Again the nanocomposite with 10 wt. % of 10 nm silicon oxide was measured to have twice the viscosity of the pure oil system. Investigation of the viscosity sweeps in FIG. 22 reveals that the nanoparticles have no influence on the pour point transition temperature of the oils. This constant pour point temperature was further demonstrated by the DSC scans where the peaks for the pure oil and the oil —SiOₓ particle nanocomposites were seen to occur at identical temperatures.

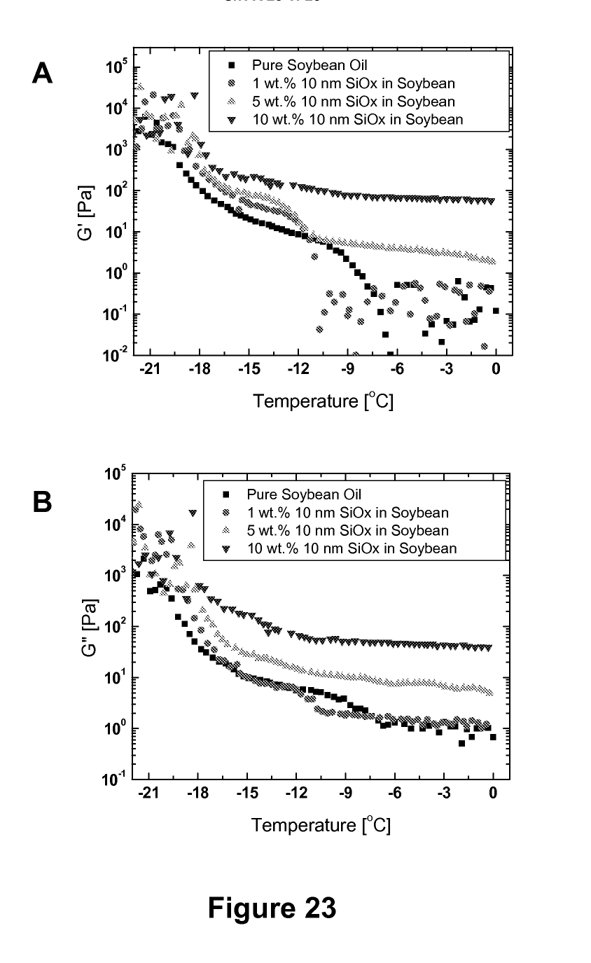

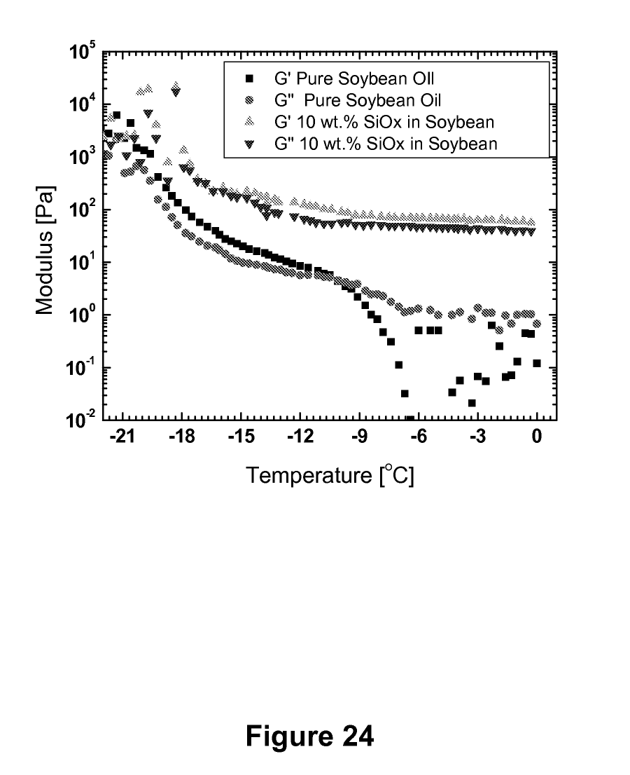

The addition of nanoparticles to vegetable oil systems also increased the mechanical properties of the oils as shown in FIG. 23. The storage modulus of a soybean oil mixture with 1 wt. % of the 10 nm diameter silicon oxide particles behaved like the pure oil at temperatures higher than the pour point regime, but G′ is roughly doubled for system temperatures below the pour point. The oil blended with 5 wt. % SiOₓ particles showed an order of magnitude increase at temperatures above the pour point and followed the same trend as the 1 w/w % solution below the pour point. The most dramatic effect on the mechanical properties occurred for the oil nanocomposite with 10 wt. % SiOₓ particles where the G′ values increased by two orders of magnitude above the pour point temperature and one order of magnitude below the pour point temperature. Similar effects were observed in the loss modulus where the 1 wt. % SiOₓ nanocomposite had similar values to the pure oil, the 5 wt. % SiOₓ solution had roughly double the G″, and the 10 wt. % SiOₓ solution showed an order of magnitude increase in G″ for all temperatures. The observed increase in G′ suggests that the system is becoming a stronger gel with the increase in the concentration of nanoparticles added. The loss tangent, tan δ, is defined as the ratio of G″/G′ and can be used to describe the nature of the material, where a high loss tangent (>>1) means a more liquid-like system and a low value (<<1) is a solid-like system. At temperatures above the pour point the soybean oil and SiOₓ systems that have been considered have loss tangents ranging from being liquid-like, e.g. loss tangents of ˜3-4 and ˜2 for the systems with 1 and 5 wt. % particles, respectively, to being solid-like with a loss tangent of ˜0.6 for the system with 10 wt. % particles. An illustration of the change in the system from the liquid-like pure oil to the more gel-like blend with 10 wt. % 10 nm SiOₓ particles can be seen in comparing the G′ and G″ of these two systems (see FIG. 24). FIGS. 2 and 24 are not the same experiment but both compare the storage and loss modules of soybean oil and soybean oil with 10 wt % 10 nm SiOₓ. In combination these figures demonstrate repeatability of increase of the moduli values. Unlike for the pure oil where G′ is less than G″ above the pour point and then crosses near the pour point temperature, G′ is always larger than G″ for the 10 wt. % system suggesting that the system is solid- or gel-like at all temperatures.

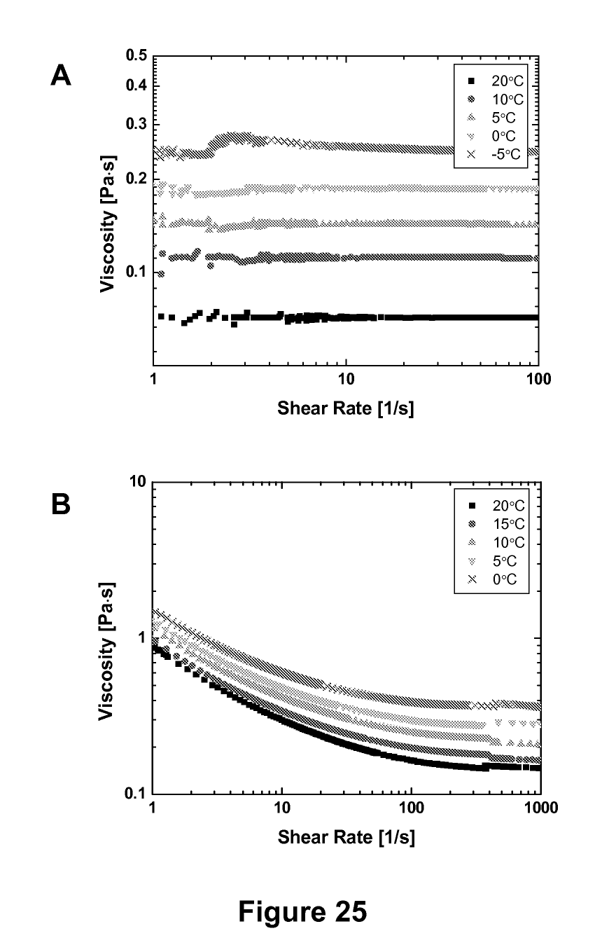

A comparison of the shear viscosity as a function of shear rate has been performed for the pure soybean oil and the soybean oil with 10 wt % 10 nm SiOₓ particles (see FIG. 25). The viscosity of the nanoparticle system decreases an order of magnitude over the shear rate range tested (1 to 1000 Hz) whereas the pure oil shows minimal shear thinning. The observed shear thinning behavior may limit the use of the nanoparticle soybean solutions as lubricants. This does not limit, however, the concept that nanoparticle additives can be used to strengthen vegetable oil-based systems. For example, soywax, which is essentially a blended soybean oil that is solid at room temperature, has been used for nearly a decade as a natural alternative to beeswax since it is much cheaper to produce.[44] It is also a promising alternative to other naturally occurring and more expensive waxes including carnauba, joyjoba, and candelilla. The major limitation of using soywax for many applications has been its less than ideal mechanical strength and hardness. We have found that the addition of 10 wt. % 10 nm SiOₓ nanoparticles can increased the hardness of soywax by greater than 50

### CONCLUSIONS

In this Example we have applied rheological experiments in order to characterize and understand the pour point transition. By examining the change in the shear viscosity as a function of temperature while cooling from high temperatures to temperatures below the pour point, we have found that the viscosity behavior of the vegetable oil systems deviates at the pour point temperature. It was found for several different vegetable oils that a crossover of G′ and G″ occurred at the pour point temperature suggesting this transition is in fact a gel transition. Further investigation has shown the phase angle is frequency independent near the pour point temperature, thereby providing additional evidence that the pour point arises due to gelation of the triglyceride molecules. The addition of hydrocarbons can greatly influence the pour point by either increasing or decreasing the transition temperature depending on the melting temperature and molecular size of the additive. The experimental methods presented here can be used to characterize not only the pour point temperature of oil systems, but can simultaneously provide information about the critical physical properties of the lubricant including viscosity.

TABLE 1

| | | | Average | |
|---|---|---|---|---|
| | Shear | Cooling | Deviation | Pour Point |
| | Stress | Rate | Temperature | Temperature |
| Type of Oil | [Pa] | [° C./min] | [° C.] | [° C.] |
| Soybean | 50 | 0.5 | −10.8 ± 0.3 | −9 |
| | 50 | 1 | −11.3 ± 0.4 | −9 |
| | 100 | 0.5 | −10.9 ± 0.5 | −9 |
| | 100 | 1 | −11.5 ± 0.6 | −9 |
| | 200 | 1 | −12.7 ± 0.4 | −9 |
| Corn | 50 | 1 | −16.3 ± 0.3 | −15 |
| Canola | 50 | 1 | −22.3 ± 0.2 | −21 |
| 50:50 | 50 | 1 | −15.2 ± 0.5 | −15 |
| Soybean:Canola | | | | |

TABLE 2

| | Shear | Cooling | Crossover | Pour Point |
|---|---|---|---|---|
| | Stress | Rate | Temperature | Temperature |
| Type of Oil | [Pa] | [° C./min] | [° C.] | [° C.] |
| Soybean | 0.1 | 0.5 | −8.4 ± 0.5 | −9 |
| | 0.1 | 1 | −9.9 ± 0.5 | −9 |
| | 0.5 | 1 | −10.3 ± 0.4 | −9 |
| | 1 | 1 | −11.9 ± 0.7 | −9 |
| Corn | 0.1 | 1 | −12.2 ± 0.7 | −15 |
| Canola | 0.1 | 1 | −23.3 ± 0.8 | −21 |
| 50:50 | 0.1 | 1 | −15.0 ± 0.6 | −15 |
| Soybean:Canola | | | | |

TABLE 3

| | | | Viscosity | |
|---|---|---|---|---|
| | Molecular | Density | at 25° C. | Melting Point |
| Hydrocarbon | Formula | [g/cm³] | [mPa · s] | [° C.] |
| Hexanes | | | 0.3 | −95 |
| n-Octane | C₈H₁₈ | 0.708 | 0.508 | −57 |
| 1-Decene | C₁₀H₂₀ | 0.741 | | −66.3 to −66 |
| n-Decane | C₁₀H₂₂ | 0.735 | 1.277 | −30 |
| n-Hexadecane | C₁₆H₃₄ | 0.77 | 3.302 | 18 |
| n-Eicosane | C₂₀H₄₂ | 0.7886 | n/a | 36-38 |

TABLE 4

| | Shear | Cooling | Average Deviation | Pour Point |
|---|---|---|---|---|
| Weight % | Stress | Rate | Temperature | Temperature |
| Hydrocarbon | [Pa] | [° C./min] | [° C.] | [° C.] |
| 5% Hexanes | 100 | 1 | −14.35 ± 0.3 | |
| 5% n-Octane | 100 | 1 | −13.89 ± 0.4 | |
| 5% 1-Decene | 100 | 1 | −14.2 ± 0.3 | −15 |
| 5% n-Decane | 100 | 1 | −13.4 ± 0.2 | −12 |
| 5% n-Hexadecane | 100 | 1 | −9.35 ± 0.4 | −9 |
| 5% n-Eicosane | 100 | 1 | 6.8 ± 0.6 | 6 |
| 10% 1-Decene | 100 | 1 | −14.7 ± 0.2 | −15 |

### STATEMENTS REGARDING INCORPORATION BY REFERENCE AND VARIATIONS

U.S. Pat. Nos. 6,797,020, issued Sep. 28, 2004, and 5,976,560, issued Nov. 2, 1999, relate to waxes derived from vegetable oils are hereby incorporated by reference in their entireties to the extent not inconsistent with the present description.

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

When a group of substituents is disclosed herein, it is understood that all individual members of that group and all subgroups, including any isomers, enantiomers, and diastereomers of the group members, are disclosed separately. When a Markush group or other grouping is used herein, all individual members of the group and all combinations and subcombinations possible of the group are intended to be individually included in the disclosure. When a compound is described herein such that a particular isomer, enantiomer or diastereomer of the compound is not specified, for example, in a formula or in a chemical name, that description is intended to include each isomers and enantiomer of the compound described individual or in any combination. Additionally, unless otherwise specified, all isotopic variants of compounds disclosed herein are intended to be encompassed by the disclosure. For example, it will be understood that any one or more hydrogens in a molecule disclosed can be replaced with deuterium or tritium. Isotopic variants of a molecule are generally useful as standards in assays for the molecule and in chemical and biological research related to the molecule or its use. Methods for making such isotopic variants are known in the art. Specific names of compounds are intended to be exemplary, as it is known that one of ordinary skill in the art can name the same compounds differently.

Many of the molecules disclosed herein contain one or more ionizable groups [groups from which a proton can be removed (e.g., —COOH) or added (e.g., amines) or which can be quaternized (e.g., amines)]. All possible ionic forms of such molecules and salts thereof are intended to be included individually in the disclosure herein. With regard to salts of the compounds herein, one of ordinary skill in the art can select from among a wide variety of available counterions those that are appropriate for preparation of salts of this invention for a given application. In specific applications, the selection of a given anion or cation for preparation of a salt may result in increased or decreased solubility of that salt.

Every formulation or combination of components described or exemplified herein can be used to practice the invention, unless otherwise stated.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In the following description use of the term “about” when modifying a number or numerical range indicates a value that may vary by a small amount, for example, by 1 percent, 2 percent, 3 percent or 5 percent. Whenever a numerical range is specific with a lower limit (R_{L}) and an upper limit (Rᵤ), any value falling within the range is specifically disclosed, such as values as defined by the expression R=(R_{L})+k*(Rᵤ−R_{L}), wherein k is a variable ranging from 1 percent to 100 percent with a 1 percent increment (i.e., k=1%, 2%, 3% . . . 50%, 51% . . . 99% or 100%).

### REFERENCES

[1] Zaher, F. A.; Nomany, H. M. Grasas Aceites 1988, 49, 235.
[2] Erhan, S. Z.; Asadauskas, S. Ind. Crops Prod. 2000, 11, 277.
[3] Willing, A. Chemosphere 2001, 43, 89.
[4] Pirro, D. M.; Wessol, A. A. Environmental Lubricants. 2nd ed; Marcel Dekker: New York, 2001, pp 105-118.
[5] Horner, D. J. Synth Lubr 2002, 18, 327.
[6] Kodali, D. R. Ind Lubr Tribol, 2002, 54, 165.
[7] Choi, U.S.; Ahn, B. G.; Kwon, O. K.; Chun, Y. J. Tribol. Int. 1197. 30, 667.
[8] Birova, A.; Pavlovicova, A.; Cvengros, J. J. Soc. Tribol. Lubr. Eng. 2002, 18, 291.
[9] Asadauskas, S.; Perez, J. M.; Duda, J. L. J. Soc. Tribol. Lubr. Eng., 1997, 35.
[10] Annual Book of ASTM Standards, D97, 1993.
[11] Ashbaugh H. S.; Guo X. H.; Schwahn D.; Prud'homme R. K.; Richter D.; Fetters L. J., Energy & Fuels, 2005, 19, 138-144.
[12] Pirro, D. M.; Wessol, A. A., Lubrication Fundamentals, Marcel Dekker Inc., New York, 2001
[13]. Smalheer, C. V.; Smith, R. K., Lubricant Additives, Lezius-Hiles Co., Cleveland, 1967.
[14] Asadauskas, S. et al. J. Am. Oil. Chem. Soc. 1999, 76, 313.
[15] Ming T. C.; Ramli N.; Lye O. T.; Said M.; Kasim Z., Euro. J. Lipid Sci. Tech., 2005, 107, 505-512.
[16] Pedersen, K. S.; Ronnignsen, H. P., Energy & Fuels, 2003, 17, 321-328.
[17] Pedersen, K. S.; Ronnignsen, H. P., Energy & Fuels, 2000, 14, 43-51.
[18] Patel, H A, Somani, R J, Bajaj, H C, Jasrva, R V, Bull. Mat. Sci., 2006, 29, 133-145.
[19] Abramovic H.; Klofutar, C., Acta Chim. Slov., 1998, 45, 69-77.
[20] Venkatesan R.: Singh P.; Fogler H. S., SPE Journal, 2002, 7, 349-352.
[21] Lopes de Silva, J.; Coutinho, J. A. P., Rheol Acta, 2004, 43, 433-441.
[22] Visintin R. F. G.; Lapasin R.; Vignati E.; D'Antona P.; Lockhart T. P., Langmuir, 2005, 21, 6240-6249.
[23] Venkatesan R.; Nagarajan N. R.; Paso K.; Yi Y. B.; Sastry A. M.; Fogler H. S., Chem. Eng. Sci., 2005, 60, 3587-3598.
[24] Webber, R. M., J. Rheol., 1999, 43, 911-931.
[25] Tiwary, D., Mehrotra, A. K., Canadian J. of Chem. Eng., 2004, 82, 162-174.
[27] Garcia-Franco C. A.; Srinivas S.; Lohse D. J.: Brant P.; Macromolecules, 2001, 34, 3115-3117.
[28] Schwittay, C.; Mours, M.; Winter, H. H., Faraday Discuss., 1995, 101, 93.
[29] Chambon F.; Winter, H. H., Polym. Bull., 1985, 13, 499.
[30] Chambon F.; Winter, H. H., J. of Rheol., 1987, 31, 683-697.
[31] Winter, H. H.; Chambon F.; J. of Rheol., 1986, 30, 367.
[32] Li B, Wang X, Liu W, Tribology Letters, 2006, 22, 79-84.
[33] Perfiliev V, Moshkovith A, Verdyan A, et al., Tribology Letters, 2006, 21, 89-93.
[34] Liu G, Li X, Lu N, et al., Tribology Letters, 2005, 18, 85-90.
[35] Bakunin, V. N.; Suslov, A. Y.; Kuzmina, G. N. et al., J. of Nanoparticle Res., 2004, 2-3, 273-284.
[36] Ye W Y, Cheng T F, Ye Q, et al. Mater. Sci. and Eng. A, 2003, 359, 82-85.
[37] Erdemir A, Tribology Int., 2005, 38, 249-256.
[38] Zhu H, Wang B, Shen J M, et al. Acta Physico-Chimica Sinica, 2006, 22, 552-556.
[39] Ash, B.; Siegel, R.; Shadeler, L., Macromolecules, 2002, 37, 1358.
[40] Berriot, J.; Montes, H; Lequeux, F.;. Lequeux, Long, D.; and Sotta, P., Macromolecules. 2002, 35, 9756.
[41] Kobayashi, M.; Rharbi, Y.; Brauge, L.; Cao, L.; Winnik, M. A., Macromolecules, 2002, 35, 7387.
[42] Ou, Y.; Yang, F.; Yu, Z., J. Polym. Sci., Part B: Polym. Phys., 1998, 36, 789.
[43] Vollenberg, P.; Heikens, D.; Polymer, 1989, 30, 1656.
[44] Rezaei, K.; Wang, T.; Johnson, L. A., J. Am. Oil Chem. Soc., 2002, 79, 1241-1247.

We claim:

1. A composition comprising:
a synthetic wax derived from one or more vegetable oils; and
a plurality of nanoparticles dispersed in said synthetic wax; said nanoparticles having an average cross-sectional dimension selected from the range of about 1 nanometer to about 100 nanometers;
wherein said nanoparticles comprise between about 1% and about 50% by mass of said composition.

2. The composition of claim 1 wherein said synthetic wax comprises a triglyceride-based wax.

3. The composition of claim 2 wherein said triglyceride-based wax comprises a triglyceride component that is greater than or equal to 20% by mass of said composition.

4. The composition of claim 2 wherein said triglyceride-based wax comprises a triglyceride component that is between 20% to 80% by mass of said composition.

5. The composition of claim 2 wherein said triglyceride-based wax is derived from one or more vegetable oils selected from the group consisting of: soy bean oil; sunflower oil, corn oil, canola oil, castor oil, cottonseed oil, peanut oil, olive oil, sunflower oil, rapeseed oil, and safflower oil.

6. The composition of claim 2 wherein said triglyceride-based wax is derived from a hydrogenated vegetable oil.

7. The composition of claim 1 wherein said nanoparticles are spherical and have an average diameter selected from the range of about 10 nanometers to about 50 nanometers.

8. The composition of claim 1 wherein said nanoparticles comprise between about 5% and about 30% by mass of said composition.

9. The composition of claim 1 wherein said nanoparticles are dispersed substantially uniformly throughout said synthetic wax.

10. The composition of claim 1 wherein said nanoparticles comprise one or more silicon-containing nanoparticles selected from the group consisting of: silica nanoparticles, silicon carbide nanoparticles, and silicon nitride nanoparticles.

11. The composition of claim 1 wherein said nanoparticles comprise one or more metal salt nanoparticles selected from the group consisting of: group 1 alkali metal hydroxide nanoparticles, group 1 alkali metal carbonate nanoparticles, group 1 alkali metal sulfate nanoparticles, group 1 alkali metal phosphate nanoparticles; group 1 alkali metal carboxylate nanoparticles, group 2 alkaline earth metal hydroxide nanoparticles, group 2 alkaline earth metal hydroxide carbonate nanoparticles, group 2 alkaline earth metal hydroxide sulfate nanoparticles, group 2 alkaline earth metal hydroxide phosphate nanoparticles; and group 2 alkaline earth metal hydroxide metal carboxylate nanoparticles.

12. The composition of claim 11 wherein said nanoparticles are Mg(OH)₂ nanoparticles.

13. The composition of claim 1 wherein said nanoparticles comprise one or more transition metal-containing nanoparticles selected from the group consisting of transition metal oxide nanoparticles, transition metal carbide nanoparticles and transition metal nitride nanoparticles.

14. The composition of claim 1 wherein said nanoparticles comprise carbon nanoparticles.

15. The composition of claim 14 wherein said carbon nanoparticles are one or more carbon nanoparticles selected from the group consisting of single walled carbon nanotubes, multiwalled carbon nanotubes, carbon nanorods, carbon nanofibers, and graphite particles.

16. The composition of claim 1 wherein said nanoparticles comprise metal nanoparticles.

17. The composition of claim 1 having a melting point temperature of about 45 degrees Celsius to about 60 degrees Celsius

18. The composition of claim 1 having a hardness 1.0 to 2.0 base HB at 298 K.

19. The composition of claim 1 comprising less than about 10% by mass of a petroleum-derived chemical component.

20. The composition of claim 1 comprising nanoparticle modified wax.

21. The composition of claim 1 comprising a water in oil emulsion wax.

22. An article of manufacture comprising the composition of claim 1 selected from the group consisting of: a candle, a coating wax, a polish for a vehicle, a cosmetic wax, a pharmaceutical wax and a sealing wax.

23. The composition of claim 1 further comprising one or more additives selected from the group consisting of: a surfactant, a colorant, a fragrance and an emulsifying agent.

24. A method for enhancing at least one mechanical property of a wax composition derived from one or more vegetable oils; said method comprising:
providing a synthetic wax derived from one or more vegetable oil; and
dispersing in said synthetic wax a plurality of nanoparticles thereby making said wax composition; said nanoparticles having an average cross-sectional dimension selected from the range of about 1 nanometers to about 100 nanometers; wherein said nanoparticles comprise between about 1% and about 50% by mass of said wax composition; thereby enhancing at least one mechanical property of said wax composition.

25. The method of claim 24 comprising a method of increasing the hardness of said wax composition.

26. The method of claim 24 comprising a method of increasing the durability of said wax composition.

27. The method of claim 24 comprising a method of increasing the solidity of said wax composition.

28. The method of claim 24 wherein said step of dispersing nanoparticles in said synthetic wax does not result in a decreasing the melting point of said synthetic wax.

29. A method of making a wax composition derived from one or more vegetable oils; said method comprising the steps of:
providing a synthetic wax derived from one or more vegetable oil; and
dispersing in said synthetic wax a plurality of nanoparticles thereby making said wax composition; said nanoparticles having an average cross-sectional dimension selected from the range of about 1 nanometers to about 100 nanometers; wherein said nanoparticles comprise between about 1% and about 50% by mass of said wax composition, thereby making said wax composition derived from one or more vegetable oils.

30. A candle comprising:
a wax composition comprising synthetic wax derived from one or more vegetable oils and a plurality of nanoparticles dispersed in said synthetic wax; said nanoparticles having an average cross-sectional dimension selected from the range of about 1 nanometer to about 100 nanometers; wherein said nanoparticles comprise between about 1% and about 50% by mass of said wax composition; and
a wick disposed in said wax composition.
